(19) United States
(12) Patent Application Publication
(10) Pub. No.: US 2025/0101021 A1
(43) Pub. Date: Mar. 27, 2025

(54) METHODS OF MAKING TOLEBRUTINIB

(71) Applicant: Principia Biopharma Inc., Bridgewater, NJ (US)

(72) Inventors: Frédéric Bailly, Saint-Bauzille-de-la-Sylve (FR); Christophe Benelli, Oraison (FR); Cyril Borie, Gignac (FR); Michaël Bosch, Marsillargues (FR); Claude Cabos, Chilly-Mazarin (FR); Alexis Charaudeau, Sisteron (FR); Laurence Janssens, Châteauneuf Val St Donat (FR); François Pacquet, Antony (FR); Fabien Rodier, Oraison (FR); Laurent Sallé, St Gély du Fesc (FR); Alfred Sodo, Mireval (FR); Sylvie Vigne, Vendargues (FR)

(73) Assignee: Principia Biopharma Inc., Bridgewater, NJ (US)

(21) Appl. No.: 18/976,520

(22) Filed: Dec. 11, 2024

Related U.S. Application Data
(63) Continuation of application No. PCT/US2023/025170, filed on Jun. 13, 2023.
(60) Provisional application No. 63/351,996, filed on Jun. 14, 2022.

(51) Int. Cl. C07D 471/04; C07D 241/24; C07D 401/12

(52) U.S. Cl. CPC C07D 471/04; C07D 241/24; C07D 401/12

(57) ABSTRACT

Disclosed herein are improved synthetic routes for making (R)-1-(1-acryloylpiperidin-3-yl)-4-amino-3-(4-phenoxyphenyl)-1 H-imidazo[4,5-c]pyridin-2(3H)-one (tolebrutinbib). Also disclosed herein are novel compounds used in the synthesis of of (R)-1-(1-acryloylpiperidin-3-yl)-4-amino-3-(4-phenoxyphenyl)-1 H-imidazo[4,5-c]pyridin-2(3H)-one.

### FIELD

Disclosed herein are methods for preparing (R)-1-(1-acryloylpiperidin-3-yl)-4-amino-3-(4-phenoxyphenyl)-1H-imidazo[4,5-c]pyridin-2(3H)-one free base (tolebrutinib), also referred to herein as a Compound of Formula (I) having the structure:

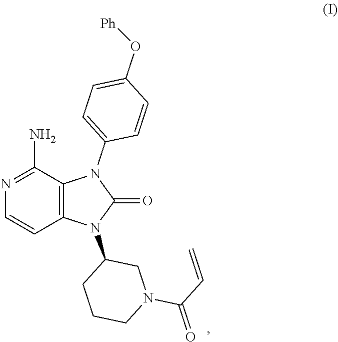 [SMILES: C=CC(=O)N1CCC[C@@H](n2c(=O)n(-c3ccc(Oc4ccccc4)cc3)c3c(N)nccc32)C1]

as well as salt forms of (R)-1-(1-acryloylpiperidin-3-yl)-4-amino-3-(4-phenoxyphenyl)-1H-imidazo[4,5-c]pyridin-2(3H)-one, and methods for preparing same. Compound (1) and its salts and solid state forms thereof are potent Bruton's Tyrosine Kinase (BTK) inhibitors and thus can be useful in the treatment of diseases or disorders resulting from an excess of BTK signaling, for example, a disease selected from an autoimmune disease, an inflammatory disease, or cancer.

### BACKGROUND AND SUMMARY

Compound (1) and a method for preparing it is disclosed in Example 3 of U.S. Pat. No. 9,688,676 B2, at column 62, line 8 to column 65 line 32, and column 67, line 28 to column 69. The disclosed synthesis provides 100 mg of crude Compound (1) that must be purified by column chromatography, resulting in 54.5 mg of purified Compound (1), which is a loss of nearly 50% of the yield. The disclosed synthesis comprises 10 steps, several of which involve undesirable solvents, and can lead to extra waste on a large scale.

One factor in the suitability of a compound as a therapeutic agent is a compound synthesis amendable to large scale manufacturing and isolation with minimal product waste and impurities. This factor is frequently considered when reviewing the suitability of a bench-scale process for making the larger quantities needed for commercial production. Moreover, the environmental impact of the various reagents and conditions needed for large scale manufacturing are an increasingly important factor.

The present disclosure relates to a method of preparing a compound of Formula (I):

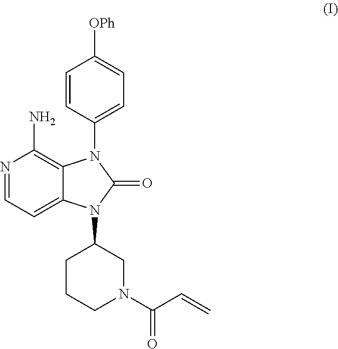 [SMILES: C=CC(=O)N1CCC[C@@H](n2c(=O)n(-c3ccc(Oc4ccccc4)cc3)c3c(N)nccc32)C1]

comprising:
reacting a compound of Formula A-oxalate:

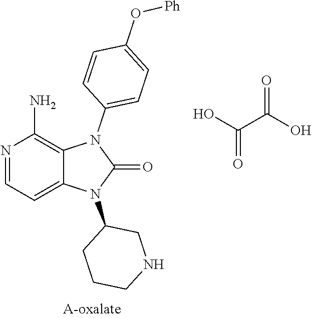 [SMILES: Nc1nccc2c1n(-c1ccc(Oc3ccccc3)cc1)c(=O)n2[C@@H]1CCCNC1 ; O=C(O)C(=O)O]

to form the compound of Formula (I), wherein the reaction conditions are chosen from:
(i) reacting the compound of Formula A-oxalate with acryloyl chloride in the presence of diisopropylethylamine in toluene; or
(ii) reacting the compound of Formula A-oxalate with 3-chloropropanoic acid in the presence of potassium carbonate, diisopropylethylamine, and propylphosphonic anhydride in dichloromethane, followed by 1,8-diazabicyclo[5.4.0]undec-7-ene in hydrochloride, and finally sodium bicarbonate.

The present disclosure also relates to a method of preparing a compound of Formula A:

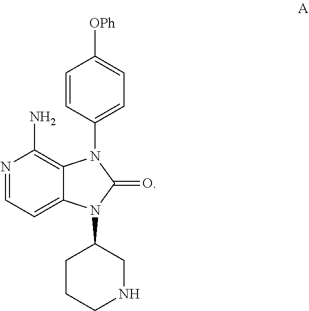 [SMILES: Nc1nccc2c1n(-c1ccc(Oc3ccccc3)cc1)c(=O)n2[C@@H]1CCCNC1]

In some embodiments, the compound of Formula A is prepared by reacting a compound of Formula 1-f:

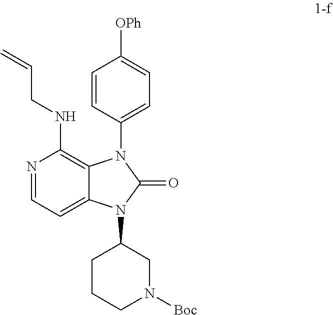 [SMILES: C=CCNc1nccc2c1n(-c1ccc(Oc3ccccc3)cc1)c(=O)n2[C@@H]1CCCN(C(=O)OC(C)(C)C)C1]

with Pd/C and methanesulfonic acid in ethanol. In some embodiments, the compound of Formula A is prepared by reacting a compound of Formula 2-d:

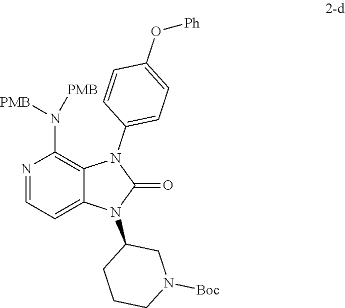

with trifluoroacetic acid in dichloromethane. In some embodiments, the compound of Formula A is prepared by reacting a compound of Formula 3-h:

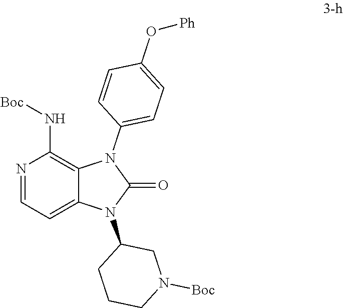 [SMILES: CC(C)(C)OC(=O)Nc1nccc2c1n(-c1ccc(Oc3ccccc3)cc1)c(=O)n2[C@@H]1CCCN(C(=O)OC(C)(C)C)C1]

with trifluoroacetic acid.

The present disclosure still further relates to a compound selected from:

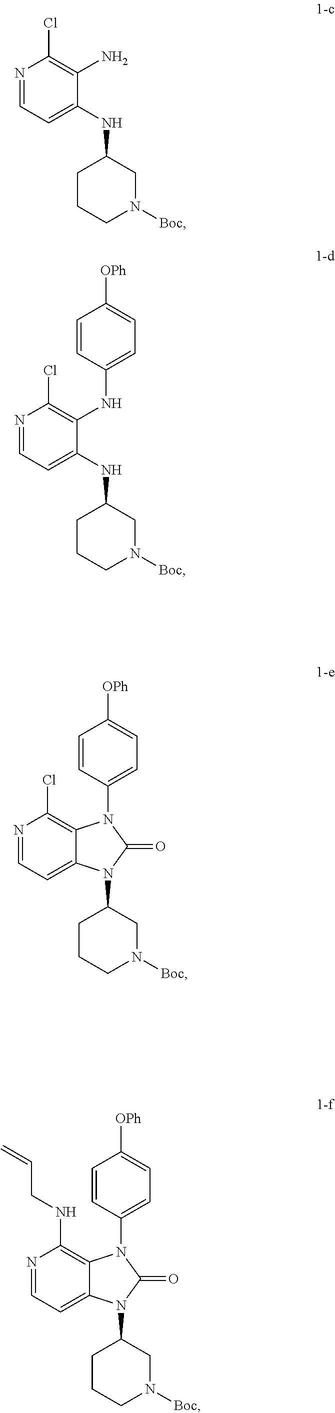 [SMILES: CC(C)(C)OC(=O)N1CCC[C@@H](Nc2ccnc(Cl)c2N)C1 ; CC(C)(C)OC(=O)N1CCC[C@@H](Nc2ccnc(Cl)c2Nc2ccc(Oc3ccccc3)cc2)C1 ; CC(C)(C)OC(=O)N1CCC[C@@H](n2c(=O)n(-c3ccc(Oc4ccccc4)cc3)c3c(Cl)nccc32)C1 ; C=CCNc1nccc2c1n(-c1ccc(Oc3ccccc3)cc1)c(=O)n2[C@@H]1CCCN(C(=O)OC(C)(C)C)C1]

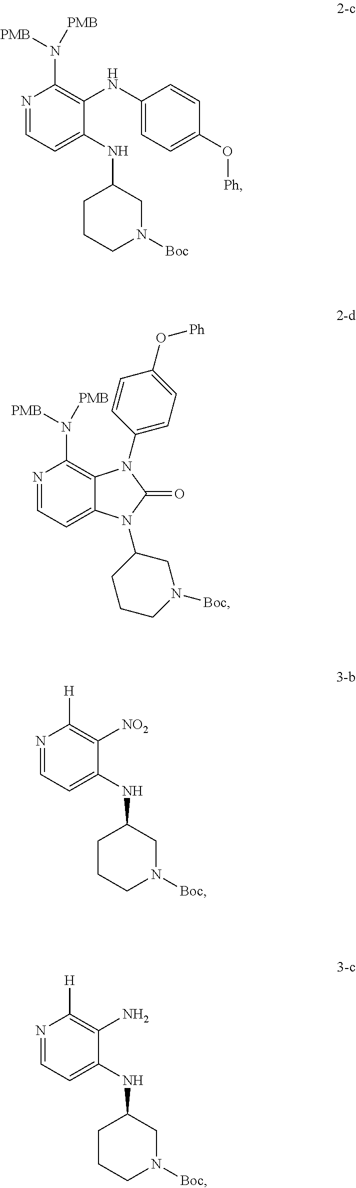

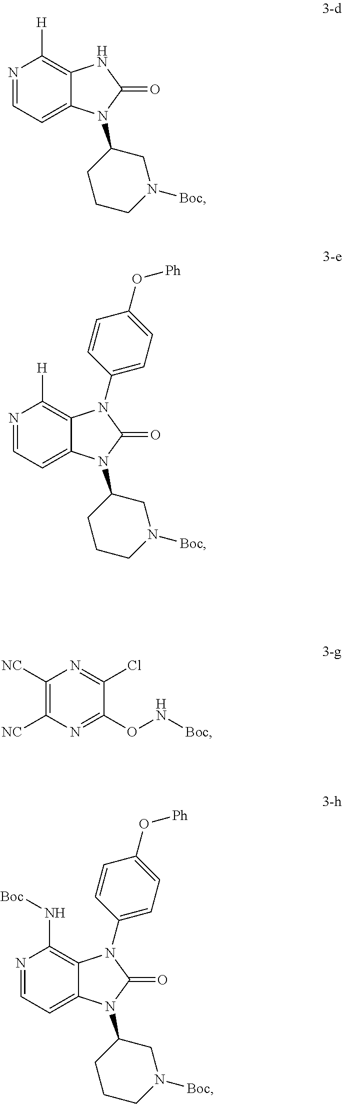 [SMILES: CC(C)(C)OC(=O)N1CCC[C@@H](n2c(=O)[nH]c3cnccc32)C1 ; CC(C)(C)OC(=O)N1CCC[C@@H](n2c(=O)n(-c3ccc(Oc4ccccc4)cc3)c3cnccc32)C1 ; CC(C)(C)OC(=O)NOc1nc(C#N)c(C#N)nc1Cl ; CC(C)(C)OC(=O)Nc1nccc2c1n(-c1ccc(Oc3ccccc3)cc1)c(=O)n2[C@@H]1CCCN(C(=O)OC(C)(C)C)C1]

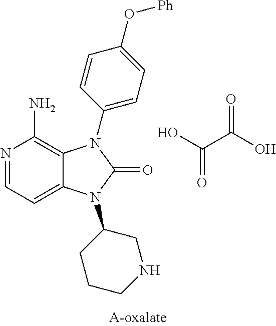

or a salt thereof.

### BRIEF DESCRIPTION OF THE DRAWINGS

FIG. 1 shows an NMR spectrum of compound 1-c.

FIG. 2 shows an NMR spectrum of compound 1-d.

FIG. 3 shows an NMR spectrum of compound 1-e.

FIG. 4 shows an NMR spectrum of compound 1-f.

FIG. 5 shows an NMR spectrum of compound 2-b.

FIG. 6 shows an NMR spectrum of compound 2-c.

FIG. 7 shows an NMR spectrum of compound A.

FIG. 8 shows a 10-step linear synthesis route for synthesizing the 2,3,4-aminopyridine ring system based on that disclosed in U.S. Pat. No. 9,688,676.

FIG. 9 shows a retrosynthetic scheme for developing improved synthesis routes.

FIG. 10 shows the comparison of a current synthesis route and a second generation synthesis route.

FIG. 11 shows an alternative synthetic strategy, wherein a route alternative to the Chan Lam coupling step is explored.

FIG. 12 shows a proof of feasibility (POF) process of the Buchwald reaction.

FIG. 13 shows a proof of feasibility (POF) process of the cyclization reaction.

FIG. 14 shows a proof of feasibility (POF) process of the deprotection reaction.

FIG. 15 shows an eco-friendly design approach of a new synthesis route.

FIG. 16 shows a comparison of a current synthesis route and another second generation synthesis route.

FIG. 17 shows a new synthesis route that avoids Chan Lam coupling through late stage NHBoc introduction.

FIG. 18 shows a CH amination step of a late stage NHBoc introduction synthesis route.

Additional objects and advantages will be set forth in part in the description which follows, and in part will be understood from the description, or may be learned by practice. The objects and advantages will be realized and attained by means of the elements and combinations particularly pointed out in the appended claims.

It is to be understood that both the foregoing general description and the following detailed description are exemplary and explanatory only and are not restrictive of the claims.

The accompanying drawings and Appendix, which are incorporated in and constitute a part of this specification, illustrate several embodiments and together with the description, serve to explain the principles described herein.

### DETAILED DESCRIPTION

### Definitions

Unless otherwise stated, the following terms used in the specification and claims are defined for the purposes of this disclosure and have the following meaning:

As used herein, “the BTK inhibitor,” “the BTK inhibitor compound,” “the compound of Formula (1),” “Compound 1,” and “the compound,” refers to (R)-1-(1-acryloylpiperidin-3-yl)-4-amino-3-(4-phenoxyphenyl)-1H- imidazo[4,5-c]pyridin-2(3H)-one having the following structure:

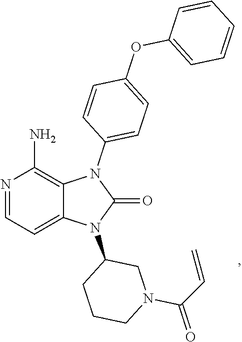 [SMILES: C=CC(=O)N1CCC[C@@H](n2c(=O)n(-c3ccc(Oc4ccccc4)cc3)c3c(N)nccc32)C1]

which is also known as 4-amino-3-(4-phenoxyphenyl)-1-[(3R)-1-(prop-2-enoyl)piperidin-3-yl]-1,3-dihydro-2H-imidazo[4,5-c]pyridin-2- one having the following structure:

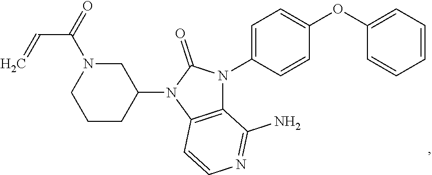 [SMILES: C=CC(=O)N1CCCC(n2c(=O)n(-c3ccc(Oc4ccccc4)cc3)c3c(N)nccc32)C1]

or a pharmaceutically acceptable salt thereof.

The present disclosure relates to a method of preparing a compound of Formula (I):

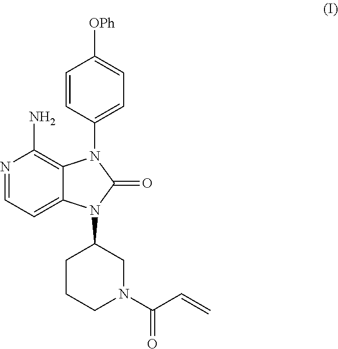 [SMILES: C=CC(=O)N1CCC[C@@H](n2c(=O)n(-c3ccc(Oc4ccccc4)cc3)c3c(N)nccc32)C1]

comprising:
reacting a compound of Formula A-oxalate:

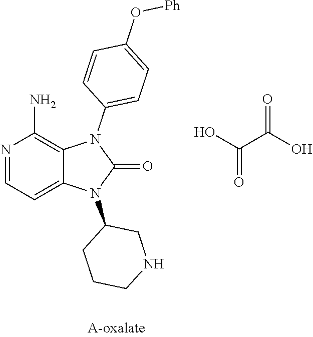 [SMILES: Nc1nccc2c1n(-c1ccc(Oc3ccccc3)cc1)c(=O)n2[C@@H]1CCCNC1 ; O=C(O)C(=O)O]

to form the compound of Formula (I), wherein the reaction conditions are chosen from:
(i) reacting the compound of Formula A-oxalate with acryloyl chloride in the presence of diisopropylethylamine in toluene; or
(ii) reacting the compound of Formula A-oxalate with 3-chloropropanoic acid in the presence of potassium carbonate, diisopropylethylamine, and propylphosphonic anhydride in dichloromethane, followed by 1,8-diazabicyclo[5.4.0]undec-7-ene in hydrochloride, and finally sodium bicarbonate.

In some embodiments, the compound of Formula A-oxalate is prepared by reacting a compound of Formula A:

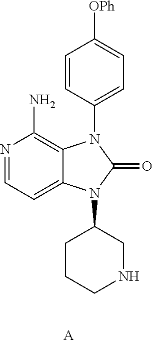

with oxalic acid in a mixture of ethanol and water.

In some embodiments, the compound of Formula A is prepared by reacting a compound of Formula 1-f:

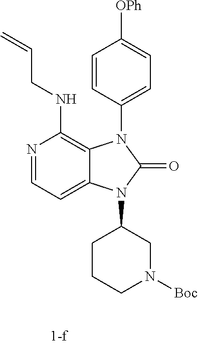 [SMILES: C=CCNc1nccc2c1n(-c1ccc(Oc3ccccc3)cc1)c(=O)n2[C@@H]1CCCN(C(=O)OC(C)(C)C)C1]

with Pd/C and methanesulfonic acid in ethanol.

In some embodiments, the compound of Formula 1-f is prepared by reacting a compound of Formula 1-e:

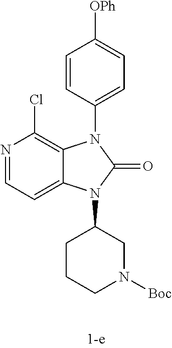 [SMILES: CC(C)(C)OC(=O)N1CCC[C@@H](n2c(=O)n(-c3ccc(Oc4ccccc4)cc3)c3c(Cl)nccc32)C1]

with allylamine in the presence of RuPhos Pd G2 and sodium tert-butoxide in dioxane.

In some embodiments, the compound of Formula 1-e is prepared by reacting a compound of Formula 1-d:

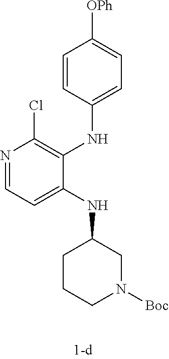 [SMILES: CC(C)(C)OC(=O)N1CCC[C@@H](Nc2ccnc(Cl)c2Nc2ccc(Oc3ccccc3)cc2)C1]

with carbonyldiimidazole in the presence of 1,8-diazabicyclo[5.4.0]undec-7-ene in toluene.

In some embodiments, the compound of Formula 1-d is prepared by reacting a compound of Formula 1-c:

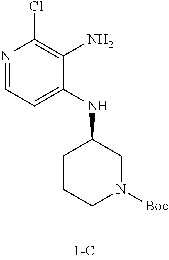 [SMILES: CC(C)(C)OC(=O)N1CCC[C@@H](Nc2ccnc(Cl)c2N)C1]

with 4-bromodiphenylether in the presence of Pd₂(dba)₃, DavePhos, and sodium tert-butoxide in toluene.

In some embodiments, the compound of Formula 1-c is prepared by reacting a compound of Formula 1-b:

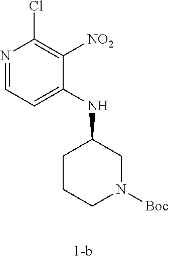 [SMILES: CC(C)(C)OC(=O)N1CCC[C@@H](Nc2ccnc(Cl)c2[N+](=O)[O-])C1]

with iron and ammonium chloride in a mixture of ethanol and water.

In some embodiments, the compound of Formula 1-b is prepared by reacting a compound of Formula 1-a:

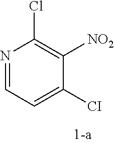

with tert-butyl (3R)-3-amino-piperidine-1-carboxylate in the presence of triethylamine in dimethylformamide.

In some embodiments, the compound of Formula A is prepared by reacting a compound of Formula 2-d:

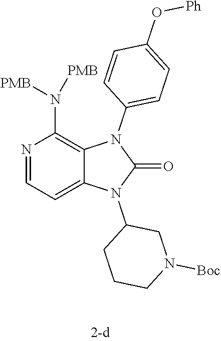

with trifluoroacetic acid in dichloromethane.

In some embodiments, the compound of Formula 2-d is prepared by reacting a compound of Formula 2-c:

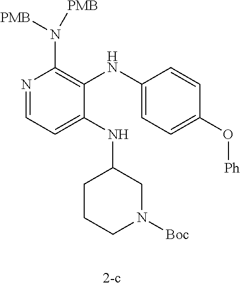

with Boc₂O in the presence of 4-dimethylaminopyridine in dimethylformamide.

In some embodiments, the compound of Formula 2-c is prepared by reacting a compound of Formula 2-b:

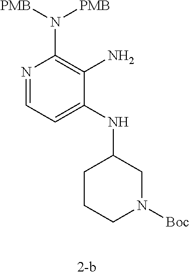

with 4-bromodiphenylether in the presence of potassium carbonate, cesium carbonate, Pd₂(dba)₃, and BrettPhos in tert-amyl methyl ether.

In some embodiments, the compound of Formula 2-b is prepared by reacting a compound of Formula 2-a:

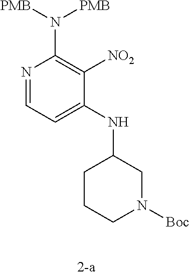

with Pd/C and H₂ in ethyl acetate.

In some embodiments, the compound of Formula 2-a is prepared by reacting a compound of Formula 1-b:

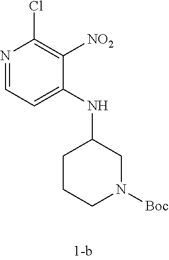 [SMILES: CC(C)(C)OC(=O)N1CCCC(Nc2ccnc(Cl)c2[N+](=O)[O-])C1]

with bis(4-methoxybenzyl)amine.

In some embodiments, the compound of Formula 1-b is prepared by reacting a compound of Formula 1-a:

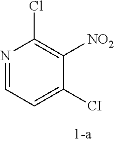

with tert-butyl (R)-3-aminopiperidine-1-carboxylate in the presence of triethylamine and HOBt in dimethylformamide.

In some embodiments, the compound of Formula A is prepared by reacting a compound of Formula 3-h:

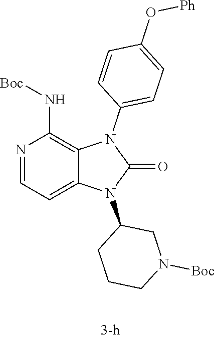 [SMILES: CC(C)(C)OC(=O)Nc1nccc2c1n(-c1ccc(Oc3ccccc3)cc1)c(=O)n2[C@@H]1CCCN(C(=O)OC(C)(C)C)C1]

with trifluoroacetic acid.

In some embodiments, the compound of Formula 3-h is prepared by reacting a compound of Formula 3-e:

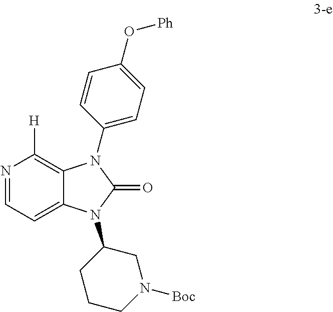 [SMILES: CC(C)(C)OC(=O)N1CCC[C@@H](n2c(=O)n(-c3ccc(Oc4ccccc4)cc3)c3cnccc32)C1]

with a compound of Formula 3-g:

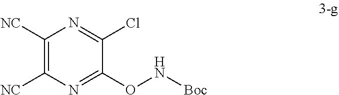 [SMILES: CC(C)(C)OC(=O)NOc1nc(C#N)c(C#N)nc1Cl]

and N,O-bis(trimethylsilyl)acetamide in dioxane, followed by zinc in acetic acid.

In some embodiments, the compound of Formula 3-g is prepared by reacting a compound of Formula 3-f:

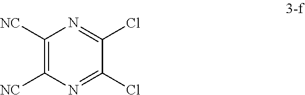 [SMILES: N#Cc1nc(Cl)c(Cl)nc1C#N]

with N-Boc-hydroxylamine in the presence of triethylamine in tetrahydrofuran.

In some embodiments, the compound of Formula 3-e is prepared by reacting a compound of Formula 3-d:

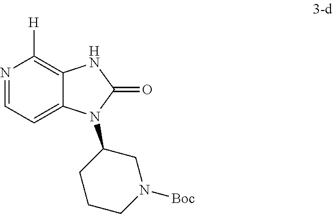 [SMILES: CC(C)(C)OC(=O)N1CCC[C@@H](n2c(=O)[nH]c3cnccc32)C1]

with 4-phenoxyphenylboronic acid in the presence of copper(II) acetate, 2,2′-bipyridine, and cesium carbonate in dichloromethane.

In some embodiments, the compound of Formula 3-d is prepared by reacting a compound of Formula 3-c:

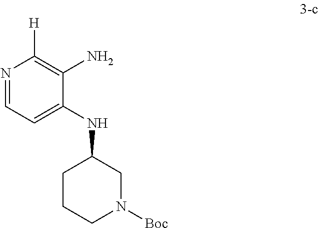 [SMILES: CC(C)(C)OC(=O)N1CCC[C@@H](Nc2ccncc2N)C1]

with carbonyldiimidazole in dimethylformamide.

In some embodiments, the compound of Formula 3-c is prepared by reacting a compound of Formula 3-b:

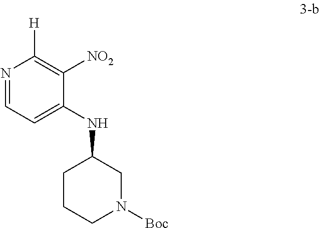 [SMILES: CC(C)(C)OC(=O)N1CCC[C@@H](Nc2ccncc2[N+](=O)[O-])C1]

with iron and ammonium chloride in ethanol.

In some embodiments, the compound of Formula 3-b is prepared by reacting a compound of Formula 3-a:

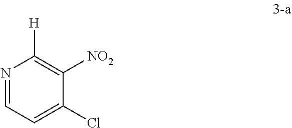

with tert-butyl (R)-3-aminopiperidine-1-carboxylate in the presence of triethylamine in dimethylformamide.

The present disclosure also relates to a compound selected from:

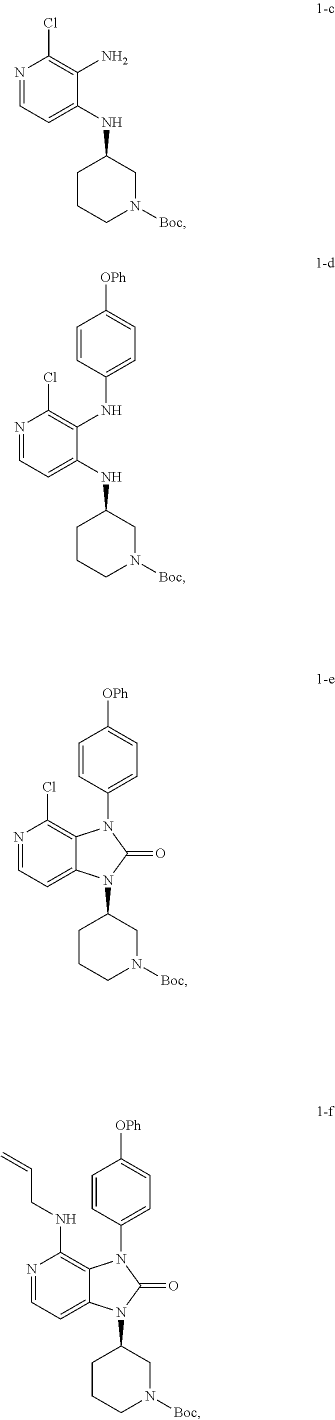 [SMILES: CC(C)(C)OC(=O)N1CCC[C@@H](Nc2ccnc(Cl)c2N)C1 ; CC(C)(C)OC(=O)N1CCC[C@@H](Nc2ccnc(Cl)c2Nc2ccc(Oc3ccccc3)cc2)C1 ; CC(C)(C)OC(=O)N1CCC[C@@H](n2c(=O)n(-c3ccc(Oc4ccccc4)cc3)c3c(Cl)nccc32)C1 ; C=CCNc1nccc2c1n(-c1ccc(Oc3ccccc3)cc1)c(=O)n2[C@@H]1CCCN(C(=O)OC(C)(C)C)C1]

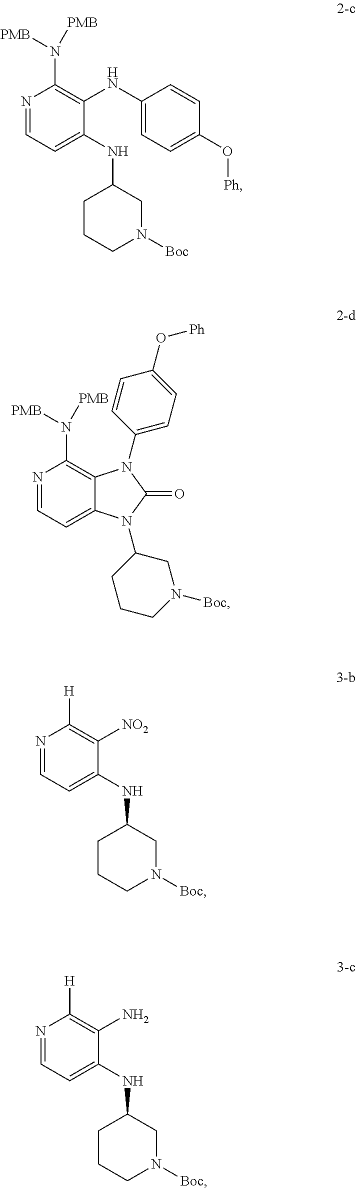

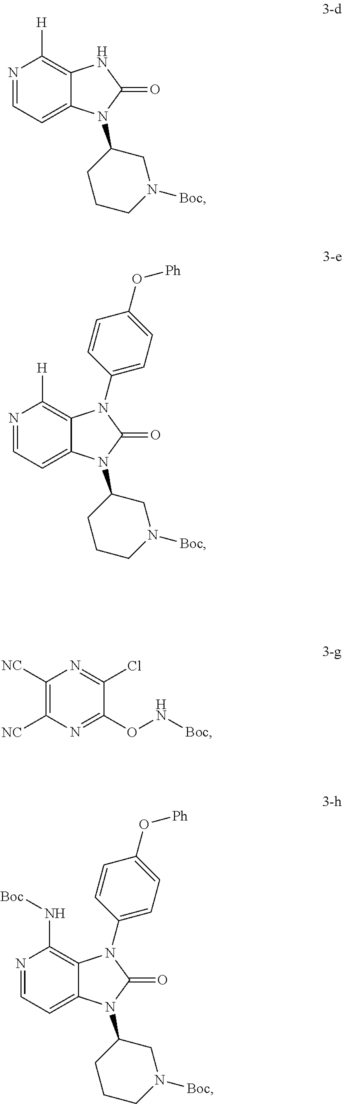 [SMILES: CC(C)(C)OC(=O)N1CCC[C@@H](n2c(=O)[nH]c3cnccc32)C1 ; CC(C)(C)OC(=O)N1CCC[C@@H](n2c(=O)n(-c3ccc(Oc4ccccc4)cc3)c3cnccc32)C1 ; CC(C)(C)OC(=O)NOc1nc(C#N)c(C#N)nc1Cl ; CC(C)(C)OC(=O)Nc1nccc2c1n(-c1ccc(Oc3ccccc3)cc1)c(=O)n2[C@@H]1CCCN(C(=O)OC(C)(C)C)C1]

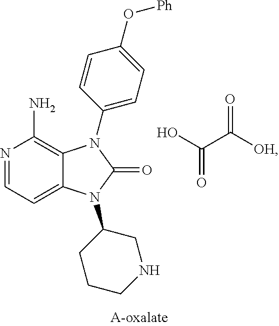

or a salt thereof.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean±20%, ±10%, ±5%, or ±1% of the indicated range, value, or structure, unless otherwise indicated.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In some embodiments, provided is a compound selected from the compounds in Table 1 or a salt thereof. Although certain compounds described in the present disclosure, including in Table 1, are presented as specific stereoisomers and/or in a non-stereochemical form, it is understood that any or all stereochemical forms, including any enantiomeric or diastereomeric forms, and any tautomers or other forms of any of the compounds of the present disclosure, including in Table 1, are herein described.

TABLE 1

| Compound No. | Structure |
|---|---|
| 1-c | 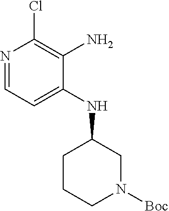 [SMILES: CC(C)(C)OC(=O)N1CCC[C@@H](Nc2ccnc(Cl)c2N)C1] |
| 1-d | 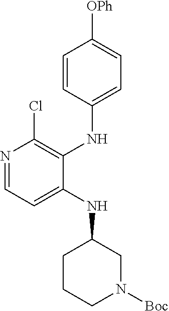 [SMILES: CC(C)(C)OC(=O)N1CCC[C@@H](Nc2ccnc(Cl)c2Nc2ccc(Oc3ccccc3)cc2)C1] |
| 1-e | 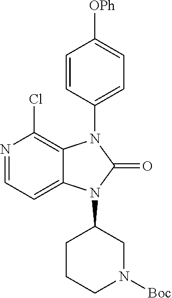 [SMILES: CC(C)(C)OC(=O)N1CCC[C@@H](n2c(=O)n(-c3ccc(Oc4ccccc4)cc3)c3c(Cl)nccc32)C1] |
| 1-f | 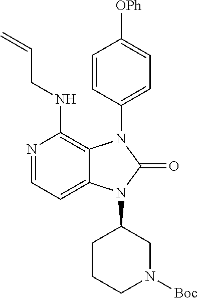 [SMILES: C=CCNc1nccc2c1n(-c1ccc(Oc3ccccc3)cc1)c(=O)n2[C@@H]1CCCN(C(=O)OC(C)(C)C)C1] |
| 2-c | 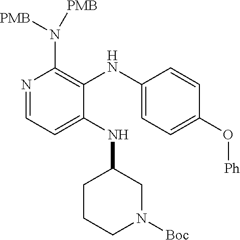 |
| 2-d | 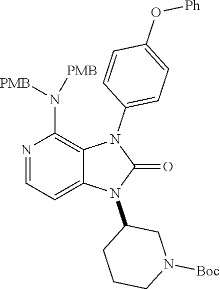 |
| 3-b | 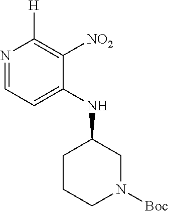 [SMILES: CC(C)(C)OC(=O)N1CCC[C@@H](Nc2ccncc2[N+](=O)[O-])C1] |
| 3-c | 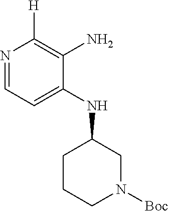 [SMILES: CC(C)(C)OC(=O)N1CCC[C@@H](Nc2ccncc2N)C1] |
| 3-d | 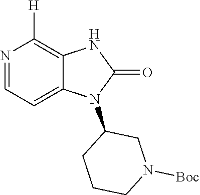 [SMILES: CC(C)(C)OC(=O)N1CCC[C@@H](n2c(=O)[nH]c3cnccc32)C1] |
| 3-e | 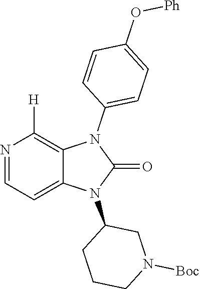 [SMILES: CC(C)(C)OC(=O)N1CCC[C@@H](n2c(=O)n(-c3ccc(Oc4ccccc4)cc3)c3cnccc32)C1] |
| 3-g | 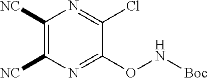 |
| 3-h | 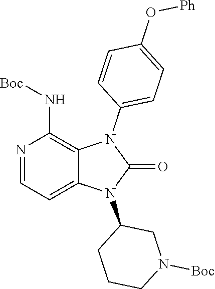 [SMILES: CC(C)(C)OC(=O)Nc1nccc2c1n(-c1ccc(Oc3ccccc3)cc1)c(=O)n2[C@@H]1CCCN(C(=O)OC(C)(C)C)C1] |
| A-oxalate | 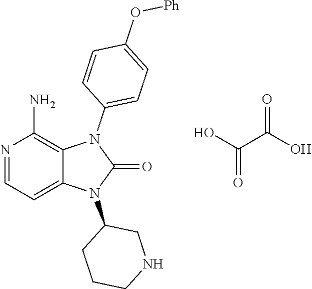 [SMILES: Nc1nccc2c1n(-c1ccc(Oc3ccccc3)cc1)c(=O)n2[C@@H]1CCCNC1 ; O=C(O)C(=O)O] |

or a salt thereof.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

2-Me-THF: 2-methyltetrahydrofuran
ACN or MeCN: acetonitrile
AcOK: potassium acetate
aq.: aqueous
BSA: bis(trimethylsilyl)acetamide
CDI: carbonyldiimidazole
d: day(s)
DBU: 1,8-diazabicyclo[5.4.0]undec-7-ene
DCM: dichloromethane
DEA: diethanolamine
DIPEA: N,N-diisopropylethylamine
DMAP: 4-dimethylaminopyridine
DMF: dimethylformamide
EA, EtOAc, or AcOEt: ethyl acetate
equiv.: equivalents
ESI: electrospray ionization
EtOH: ethanol
h or hr: hour(s)
Hex: hexanes
HOBt: hydroxybenzotriazole
HPLC: high-performance liquid chromatography
LCMS: liquid chromatography mass spectrometry
MeOH: methanol
MTBE: methyl tert-butyl ether
PE: petroleum ether
sat.: saturated
TAME: tert-amyl methyl ether
TBSOTf: tert-butyldimethylsilyl triflate
TEA: triethylamine
TFA: trifluoroacetic acid
THF: tetrahydrofuran
TLC: thin layer chromatography
V: volume per starting material (e.g., 10V solvent for 1 g of starting material=10 mL)

### Example 1

### General Route 1

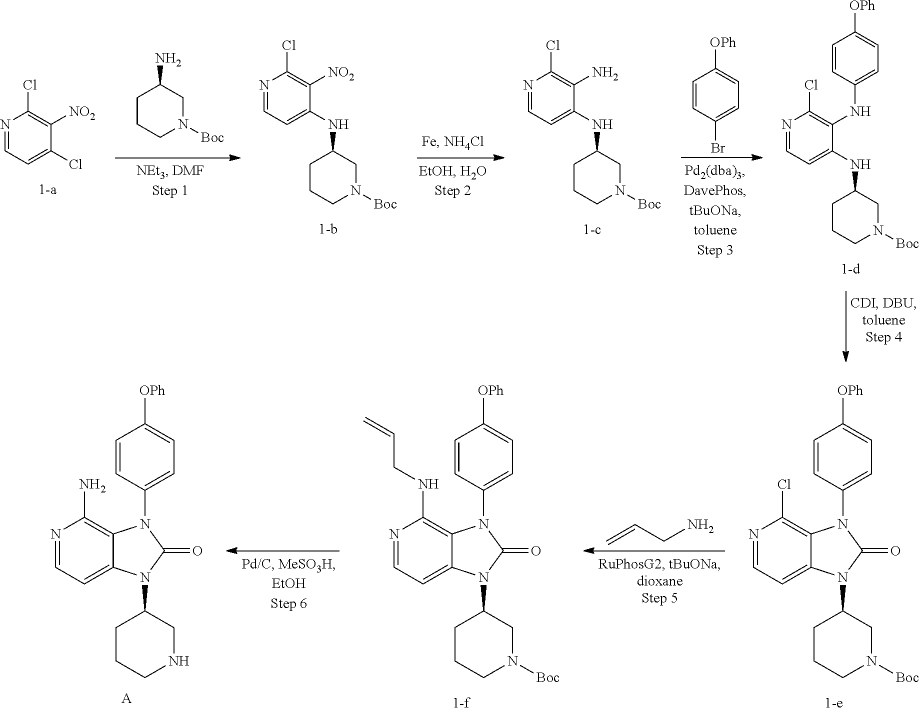

### General Route A-Example A

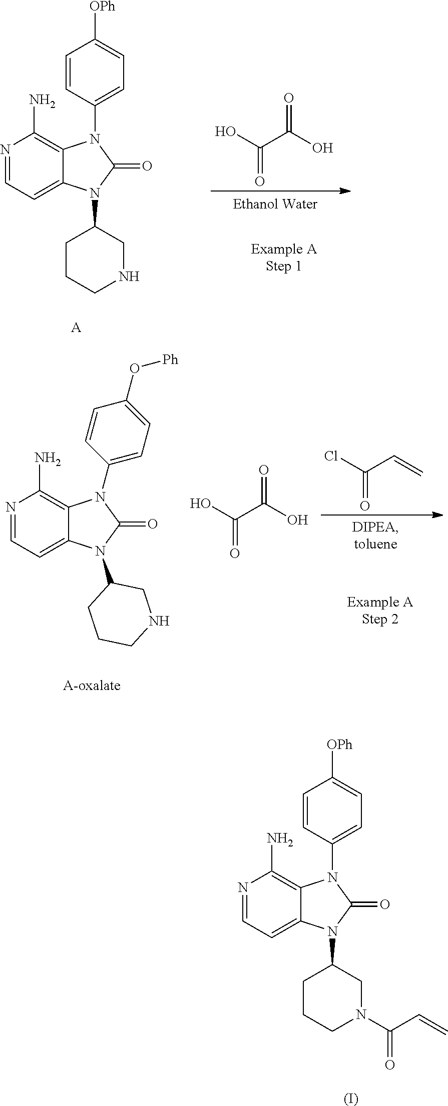

### Example S1.1 tert-butyl (R)-3-((2-chloro-3-nitropyridin-4-yl)amino)piperidine-1-carboxylate (1-b)

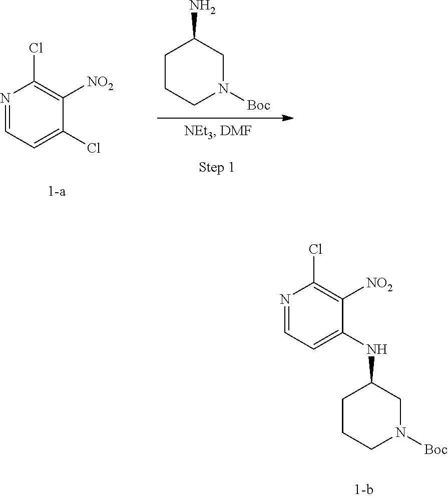 [SMILES: O=[N+]([O-])c1c(Cl)ccnc1Cl ; CC(C)(C)OC(=O)N1CCC[C@@H](N)C1 ; CC(C)(C)OC(=O)N1CCC[C@@H](Nc2ccnc(Cl)c2[N+](=O)[O-])C1]

To a solution of 2,4-dichloro-3-nitropyridine (20 g, 0.10 mol) in DMF (100 mL) was added tert-butyl (3R)-3-amino-piperidine-1-carboxylate (20.76 g, 0.10 mol) in DMF (100 mL) at 0° C. The mixture was stirred at 0° C. for 1 hr, then stirred at 20° C. for 12 h. TLC (CH₂Cl₂/Ethyl acetate=8/2) showed the complete conversion of the starting material. LCMS (ET44615-8-P1A1) showed the desired mass was detected. The aqueous phase was extracted with ethyl acetate (200 mL*2). The combined organic phase was washed with water, dried with anhydrous Na₂SO₄, filtered and concentrated in vacuum. The crude product was purified by column chromatography (SiO₂, CH₂Cl₂/Ethyl acetate=8/2). Compound 1-b (24 g, 67 mmol, 65% yield) was obtained as a yellow solid.

NMR and LCMS data were consistent with compound 1-b.

### Example S1.2 tert-butyl (R)-3-((3-amino-2-chloropyridin-4-yl)amino)piperidine-1-carboxylate (1-c)

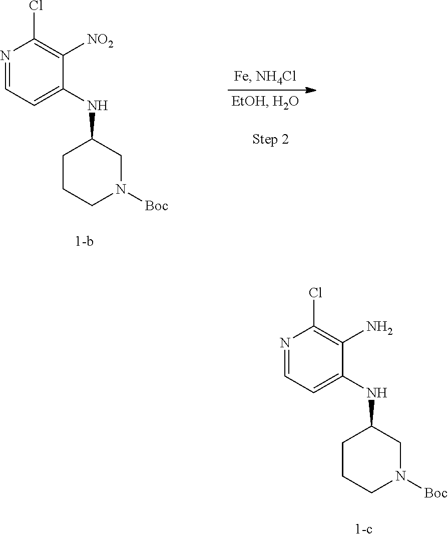 [SMILES: CC(C)(C)OC(=O)N1CCC[C@@H](Nc2ccnc(Cl)c2[N+](=O)[O-])C1 ; CC(C)(C)OC(=O)N1CCC[C@@H](Nc2ccnc(Cl)c2N)C1]

To a mixture of EtOH (64 mL) and H₂O (16 mL) was added compound 1-b (20 g, 56 mmol), NH₄Cl (14.99 g, 0.28 mol), and Fe (15.65 g, 0.28 mol, 5.00 eq) was added. The mixture was stirred at 70° C. for 4 h. LCMS analysis confirmed the complete conversion of the starting material. The mixture was filtered on celite and the residue was poured into water and extracted with ethyl acetate. The combined organic phase was washed with brine, dried with anhydrous Na₂SO₄, filtered and concentrated in vacuum. Compound 1-c (19.3 g, 56 mmol, 100% yield) was obtained as a light brown solid.

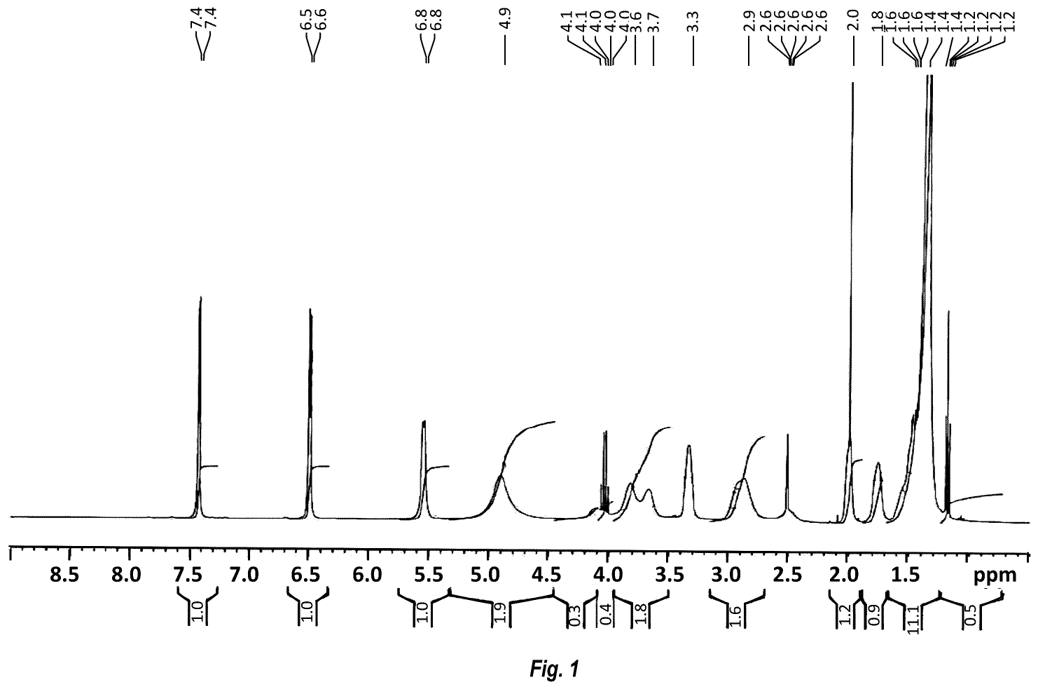

NMR data obtained was consistent with the spectrum shown in FIG. 1.

LCMS data was consistent with compound 1-c.

### Example S1.3 tert-butyl (R)-3-((2-chloro-3-((4-phenoxyphenyl)amino)pyridin-4-yl)amino)piperidine-1-carboxylate (1-d)

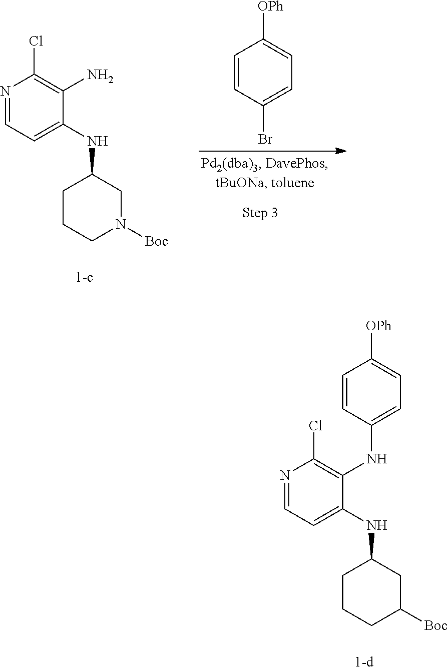 [SMILES: CC(C)(C)OC(=O)N1CCC[C@@H](Nc2ccnc(Cl)c2N)C1 ; Brc1ccc(Oc2ccccc2)cc1 ; CC(C)(C)OC(=O)C1CCC[C@@H](Nc2ccnc(Cl)c2Nc2ccc(Oc3ccccc3)cc2)C1]

Compound 1-c (6.3 g, 19 mmol) and 4-bromodiphenylether (5.07 mL, 29 mmol) were dissolved in toluene (126 mL). DavePhos (759 mg, 1.93 mmol) and Pd2 (dba) 3 (883 mg, 0.96 mmol) was added to the solution followed by sodium tert-butoxide (3.71 g, 0.39 mol). The mixture was stirred at 100° C. for 17 h. LCMS analysis confirmed the complete conversion of the starting material. The mixture was filtered on celite and the organic layer was washed with water. The solvent was removed under vacuum and the product was purified by column chromatography (SiO₂, CH₂Cl₂/Ethyl acetate=100/0 to 80/20) to obtain Compound 1-d (3.60 g, 7.3 mmol, 38% yield) as a light brown solid.

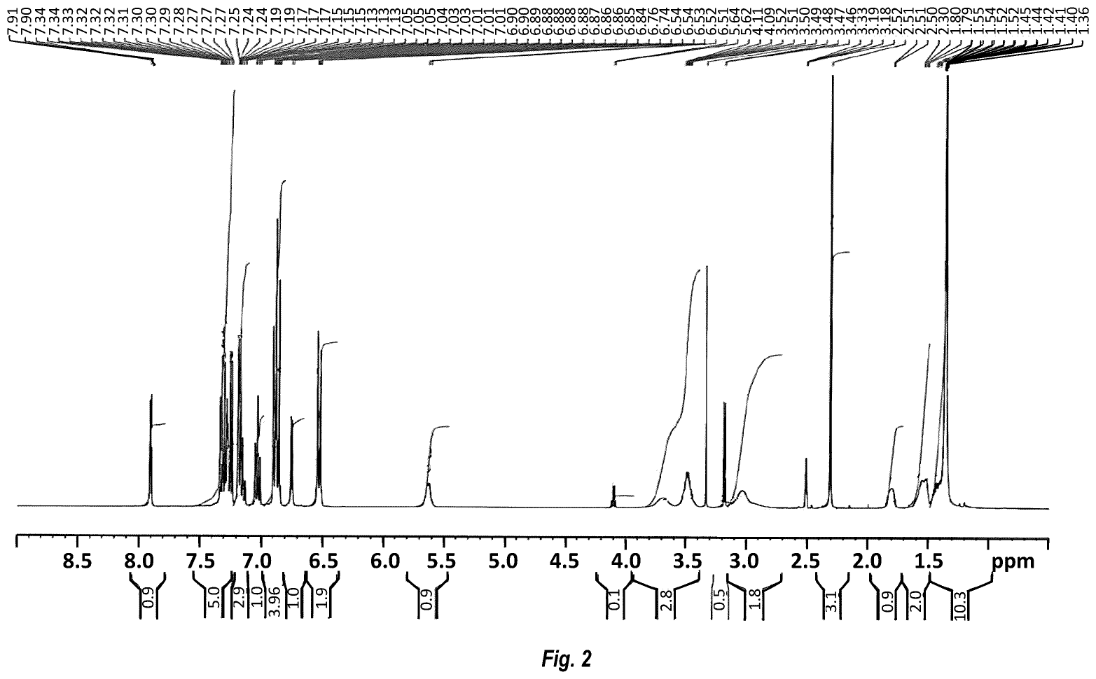

NMR data was obtained as described in FIG. 2.

LCMS data was consistent with compound 1-d.

### Example S1.4. tert-butyl (R)-3-(4-chloro-2-oxo-3-(4-phenoxyphenyl)-2,3-dihydro-1H-imidazo[4,5-c]pyridin-1-yl)piperidine-1-carboxylate (1-e)

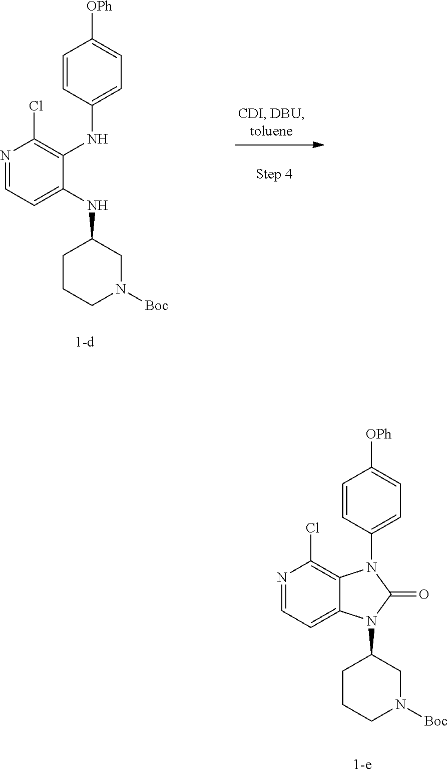 [SMILES: CC(C)(C)OC(=O)N1CCC[C@@H](Nc2ccnc(Cl)c2Nc2ccc(Oc3ccccc3)cc2)C1 ; CC(C)(C)OC(=O)N1CCC[C@@H](n2c(=O)n(-c3ccc(Oc4ccccc4)cc3)c3c(Cl)nccc32)C1]

To a mixture of compound 1-d (3.60 g, 7.27 mmol) and 1,1′-carbonyldiimidazole (2.36 g, 14.55 mmol) in toluene (36 mL) was added 1,8-diazabicyclo[5,4,0]undec-7-ene (1.09 mL, 7.27 mmol). The mixture was stirred at 100° C. for 4 h. TLC analysis (CH₂Cl₂/Ethyl acetate=8/2) confirmed the complete conversion of the starting material. The residue was poured into water (40 mL) and the organic layer was separated and washed with water until pH=7. The solvent was removed under vacuum and compound 1-e (3.2 g, 6.1 mmol, 84.5% yield) was obtained as a light brown solid.

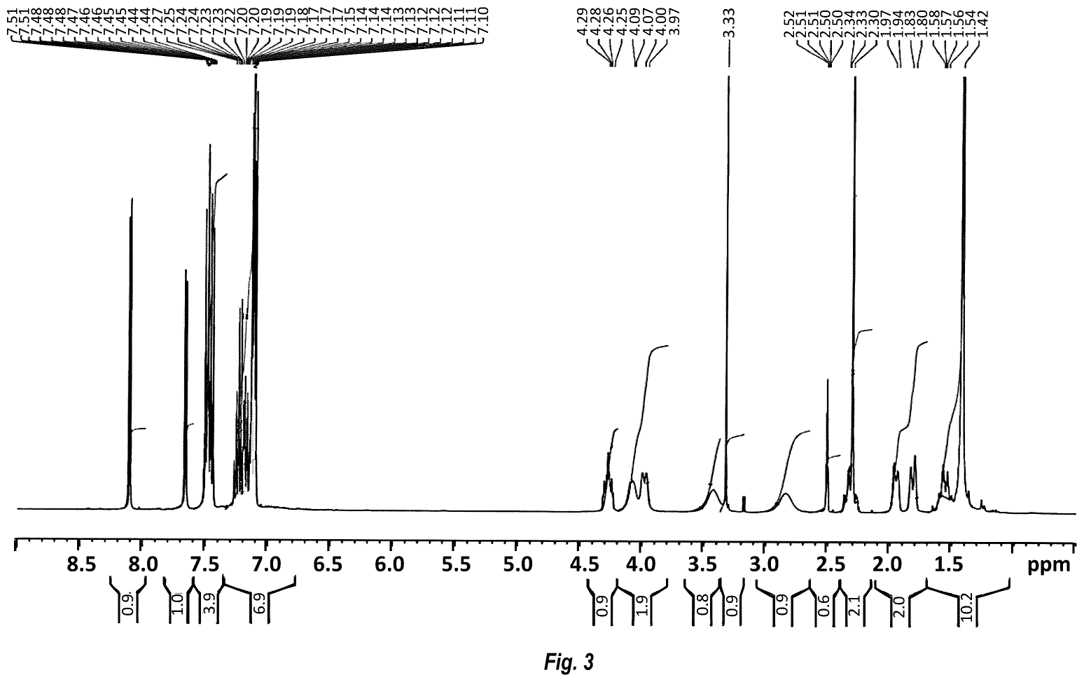

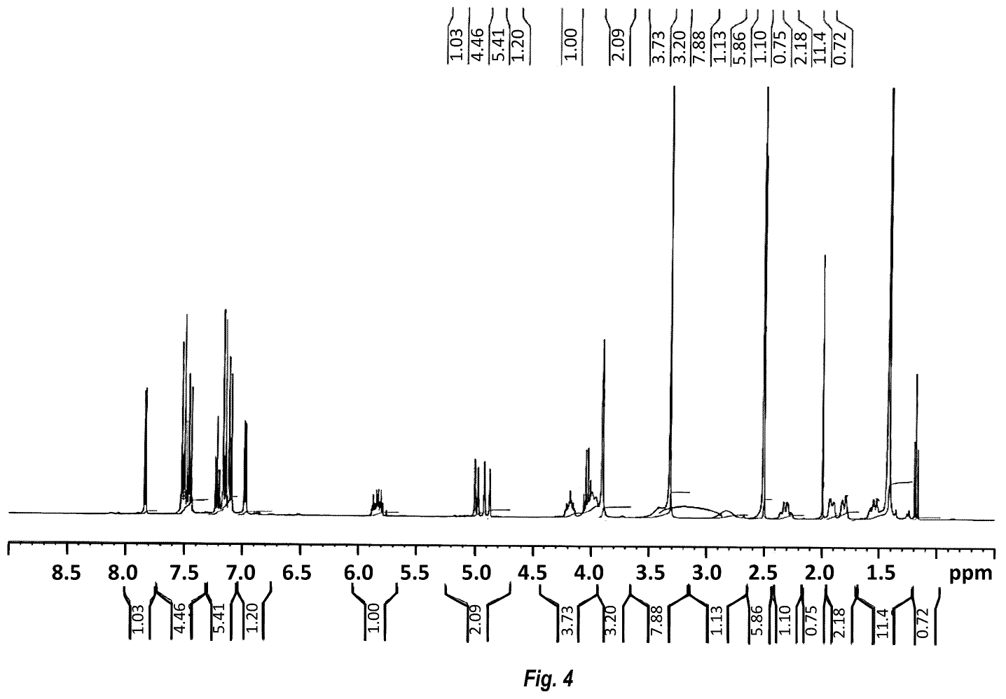

NMR data was obtained as described in FIG. 3.

LCMS data was consistent with compound 1-e.

### Example S1.5. tert-butyl (R)-3-(4-(allylamino)-2-oxo-3-(4-phenoxyphenyl)-2,3-dihydro-1H-imidazo[4,5-c]pyridin-1-yl)piperidine-1-carboxylate (1-f)

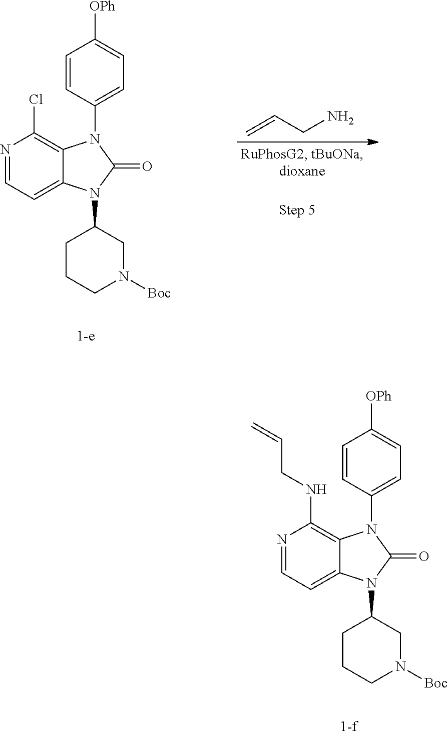

To a mixture of compound 1-e (1 g, 1.92 mmol) and allylamine (6.49 mL, 86.4mmol) in 1,4-dioxane (20 V) was added RuPhosG2 (149 mg, 0.19 mmol) and sodium tert-butoxide (276.7 mg, 2.88 mmol). The solution was sealed in a tube and stirred at 80° C. for 24 h. The mixture was filtered on celite and solvent was removed under vacuum. The residue was dissolved in ethyl acetate and water, the organic layer was separated, and the crude solid was purified by column chromatography (SiO₂, CH₂Cl₂/Ethyl acetate=100/0 to 80/20) and compound 1-f (630 mg, 1.2 mmol, 61% yield) was isolated as yellow oil.

NMR data was obtained as described in FIG. 4.

LCMS data was consistent with compound 1-f.

### Example S1.6. tert-butyl (R)-3-(4-amino-2-oxo-3-(4-phenoxyphenyl)-2,3-dihydro-1H-imidazo[4,5-c]pyridin-1-yl) piperidine-1-carboxylate (A)

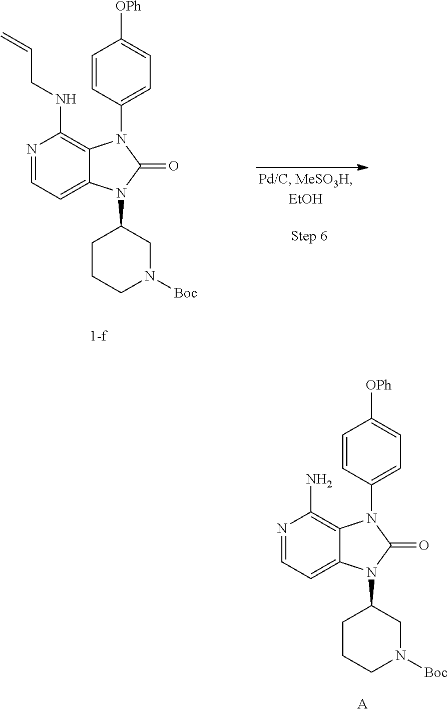

To a mixture of compound 1-f (700 mg, 1.3 mmol) and Pd/C 10% (60 mg) in ethanol (35 mL) was added methane sulfonic acid (84 μL, 1.29 mmol). The solution was stirred at 80° C. for 17 h. LCMS analysis (λ=220 nm) confirmed the complete conversion of the starting material. The mixture was filtered on celite and solvent was removed under vacuum. The residue was dissolved in ethyl acetate and water, and after basification with NaOH, the organic layer was separated. The crude product was purified by column chromatography (SiO₂, CH₂Cl₂/Ethyl acetate=100/0 to 80/20) and compound A (200 mg, 0.5 mmol, 38% yield) was isolated as a beige solid.

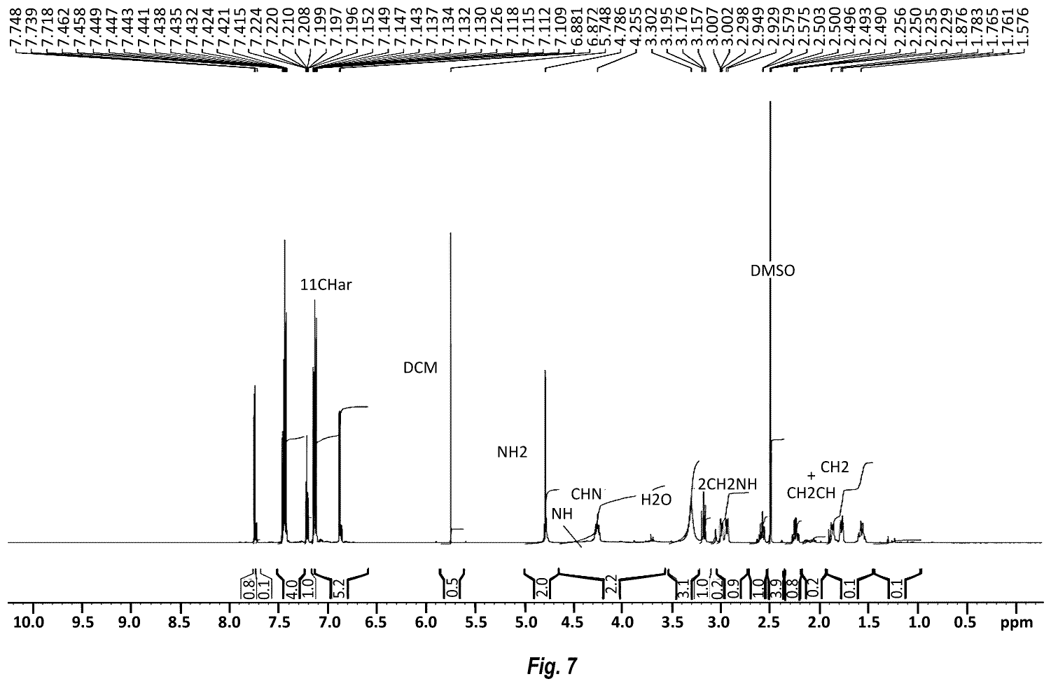

NMR data obtained was consistent with the spectrum shown in FIG. 7.

LCMS data was consistent with compound A.

### Example S1.7. (R)-1-(1-acryloylpiperidin-3-yl)-4-amino-3-(4-phenoxyphenyl)-1,3-dihydro-2H-imidazo[4,5-c]pyridin-2-one ((I))

The compound of Formula (I) was prepared following the procedure described in Example A. It could also be prepared according to the procedure described in Example B.

### Example 2

### General Route 2

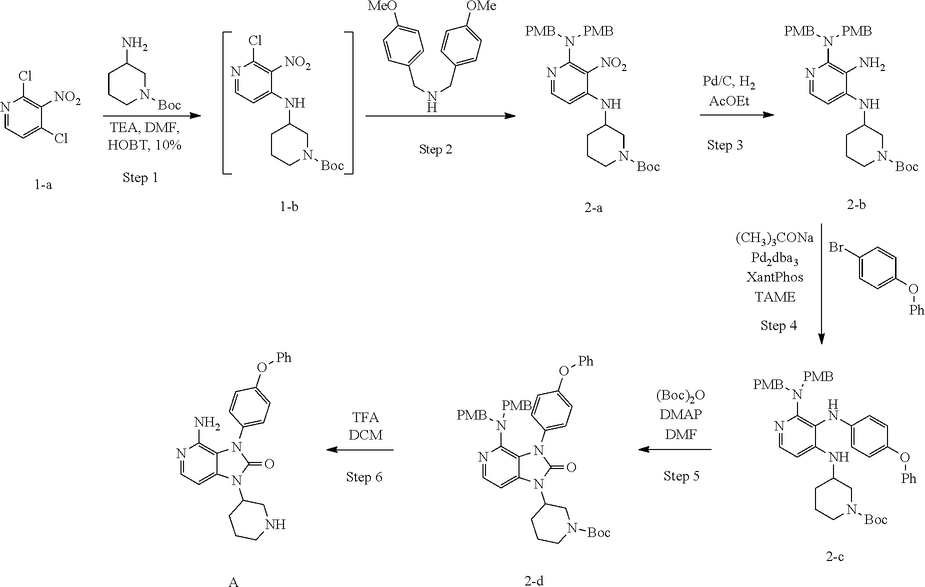

### General Route B-Example B

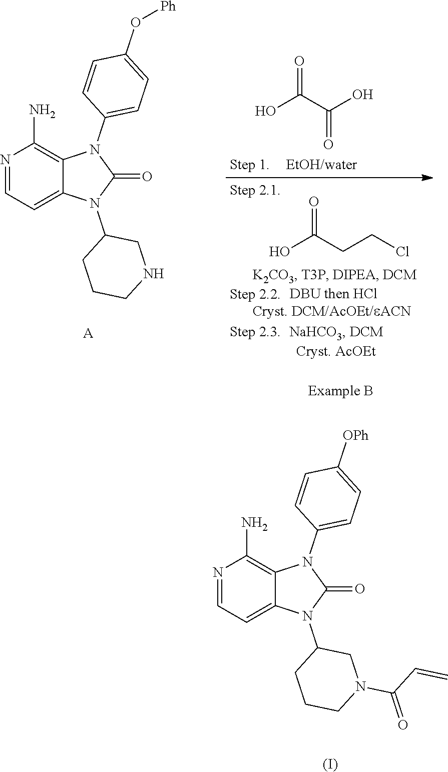

### Example S2.1 tert-butyl (R)-3-((2-chloro-3-nitropyridin-4-yl)amino)piperidine-1-carboxylate (1-b)

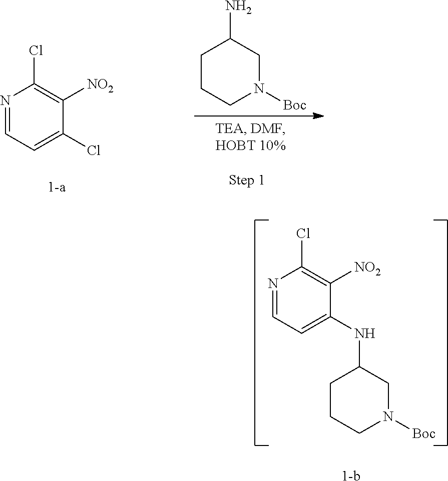

Compound 1-b can be made similar to as disclosed in U.S. Pat. No. 9,688,676. For example, compound 1-b can be prepared optionally in the presence of a HOBt catalyst.

NMR and LCMS data were consistent with compound 1-b.

### Example S2.2 tert-butyl (R)-3-((2-(bis(4-methoxybenzyl)amino)-3-nitropyridin-4-yl)amino)piperidine-1-carboxylate (2-a)

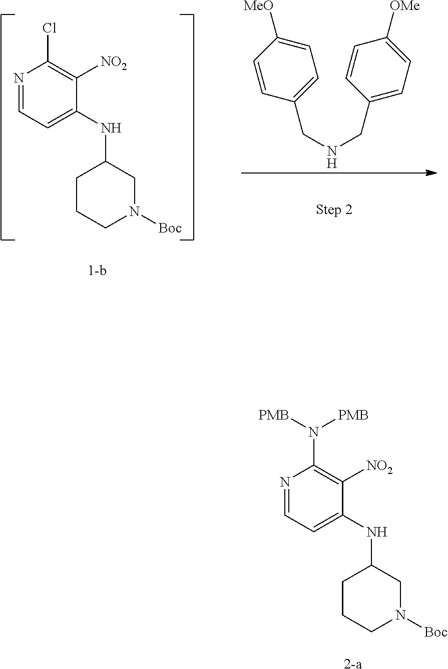

Compound 1-b can be made similar to as disclosed in U.S. Pat. No. 9,688,676.

NMR and LCMS data were consistent with compound 2-a.

### Example S2.3 tert-butyl (R)-3-((3-amino-2-(bis(4-methoxybenzyl)amino)pyridin-4-yl)amino)piperidine-1-carboxylate (2-b)

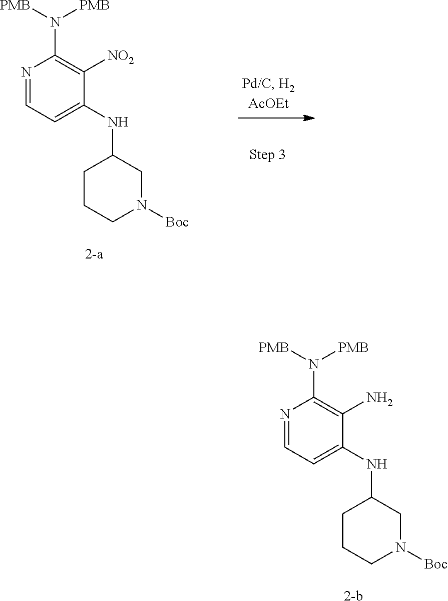

Compound 2-b can be made similar to as disclosed in U.S. Pat. No. 9,688,676. For example, compound 2-b can be prepared optionally using Pd/C and H₂ in AcOEt instead of Fe in AcOH/MeOH as described in U.S. Pat. No. 9,688,676.

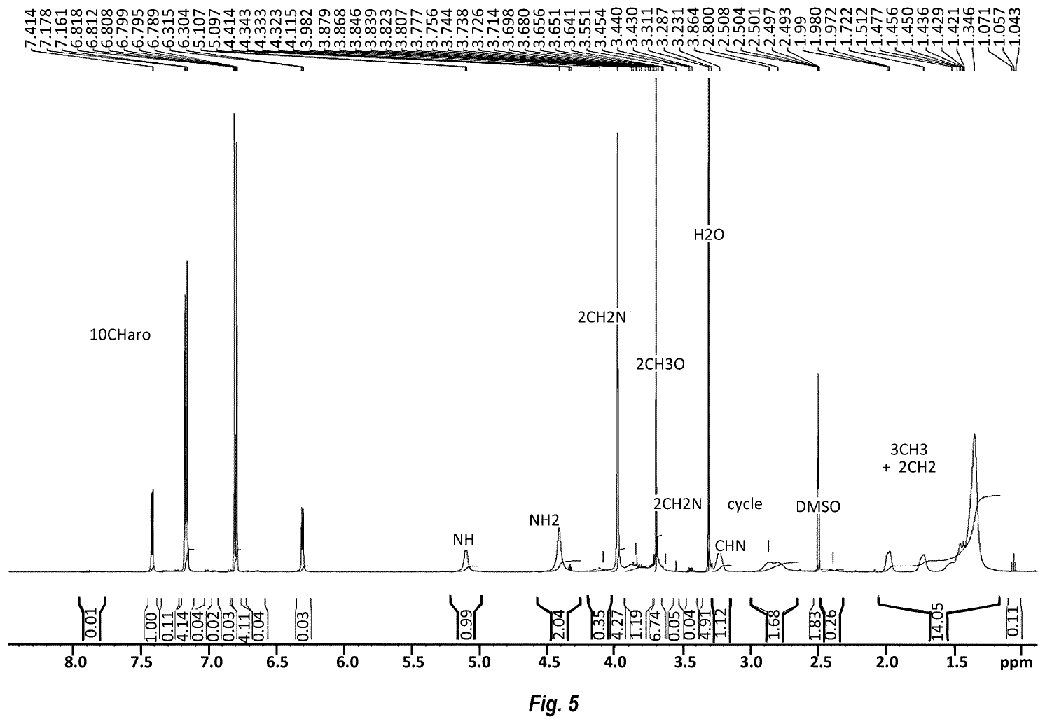

NMR data was obtained as described in FIG. 5.

LCMS data was consistent with compound 2-b.

### Example S2.4 tert-butyl (R)-3-((2-(bis(4-methoxybenzyl)amino)-3-((4-phenoxyphenyl)amino)pyridin-4-yl)amino)piperidine-1-carboxylate (2-c)

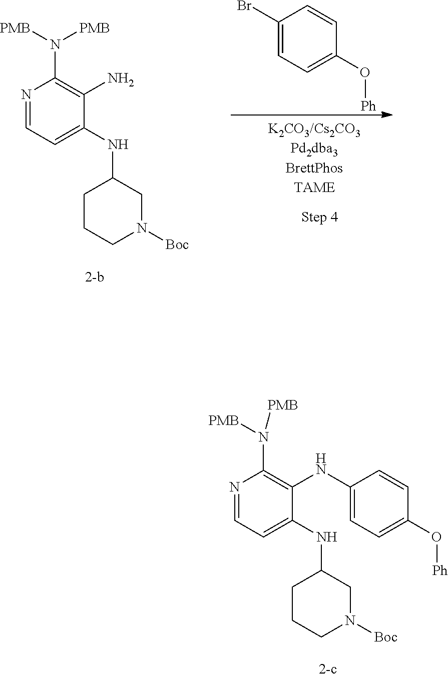

To a solution of compound 2-b (8.0 g, 14.6 mmol) in degassed tert-amylmethylether (TAME, 5V) under an inert nitrogen atmosphere were added 4-bromodiphenylether (3.07 mL, 1.2 eq), Pd₂dba₃ (669 mg, 5 mol %), BrettPhos (784 mg, 10 mol %), Cs₂CO₃ (1.43 g, 0.3 eq) and K₂CO₃ (5.45 g, 2.7 eq). The mixture was stirred at reflux for 64 h to reach 96.5% conversion by HPLC, then cooled to ambient temperature. Aqueous K₂CO₃ solution was added and the mixture was extracted by 3*10V AcOEt. Organic phases were combined and washed with 2*10V aq. K₂CO₃, 2*10V H₂O and 1*10V brine, dried on Na₂SO₄ and concentrated under reduced pressure to give 13.4 g of compound 2-c, engaged directly into next step.

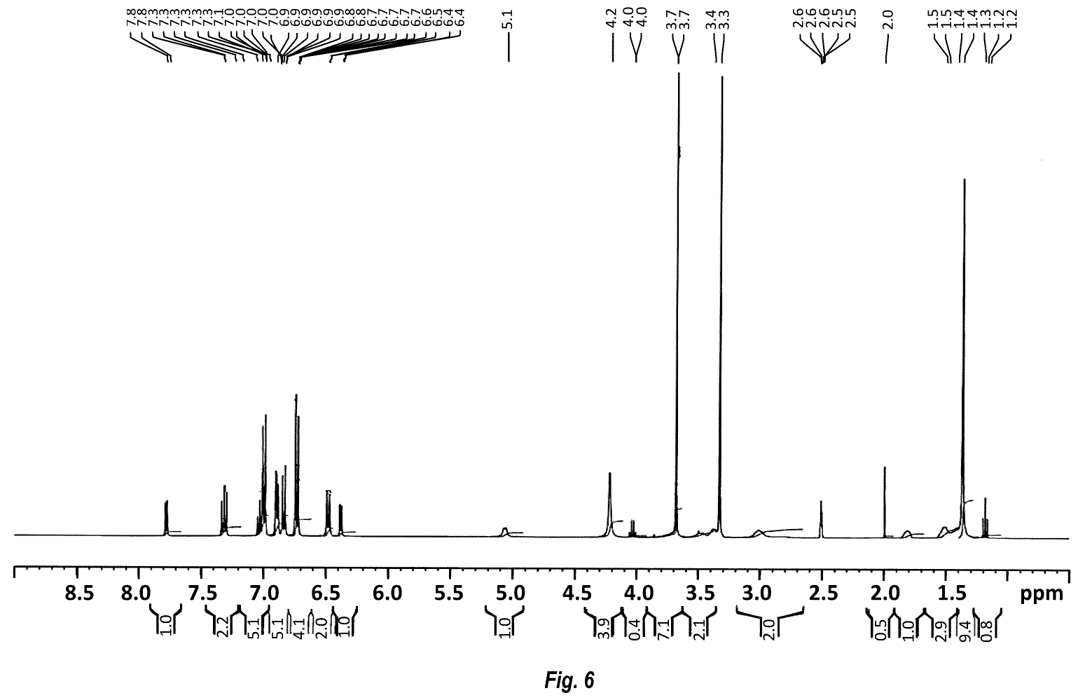

NMR data was obtained as described in FIG. 6.

LCMS data was consistent with compound 2-c.

### Example S2.5 tert-butyl (R)-3-(4-(bis(4-methoxybenzyl)amino)-2-oxo-3-(4-phenoxyphenyl)-2,3-dihydro-1H-imidazo[4,5-c]pyridin-1- yl)piperidine-1-carboxylate (2-d)

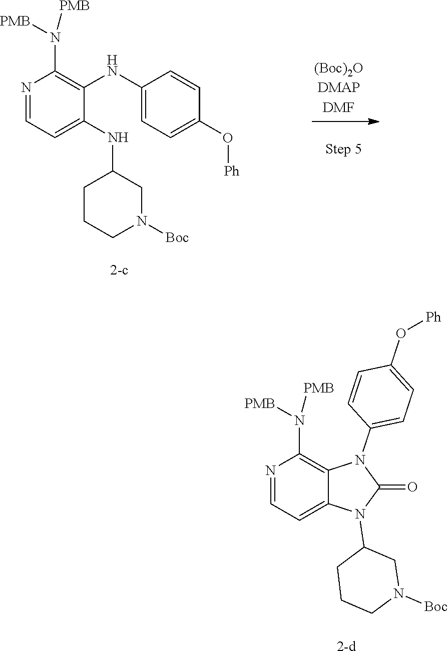

(Boc)₂O in ACN was used to give the expected compound after purification by silica gel chromatography; further laboratory trials enabled its isolation from DMF by precipitation after water addition. The reagent and solvent choice, along with the isolation conditions should be optimized before a potential multi-kilogram synthesis. Protocol: To a solution of compound 2-c (14.6 mmol) in DMF (31.4 mL, 3V) were added (Boc)₂O (7.02 g, 2.2 eq) and DMAP (178.5 mg, 10 mol %). The mixture was stirred at 45° C. for 4 h, when TLC indicated complete conversion. Mixture was cooled to 0° C. and 6V H₂O were added; the solid was filtrated, re-dissolved in 3V DMF; 6V H₂O were added at 0° C., solid was filtrated and dried under vacuum, re-dissolved in 5V DMF, and added to 15V H₂O at 0° C. Solid was filtrated, rinsed with 2*5V H₂O and dried under vacuum to give 11 g of compound 2-d (70.8% purity, 71% corrected yield), engaged directly into next step.

NMR and LCMS data were consistent with compound 2-d.

### Example S2.6 (R)-4-amino-3-(4-phenoxyphenyl)-1-(piperidin-3-yl)-1,3-dihydro-2H-imidazo[4,5-c]pyridin-2-one (A)

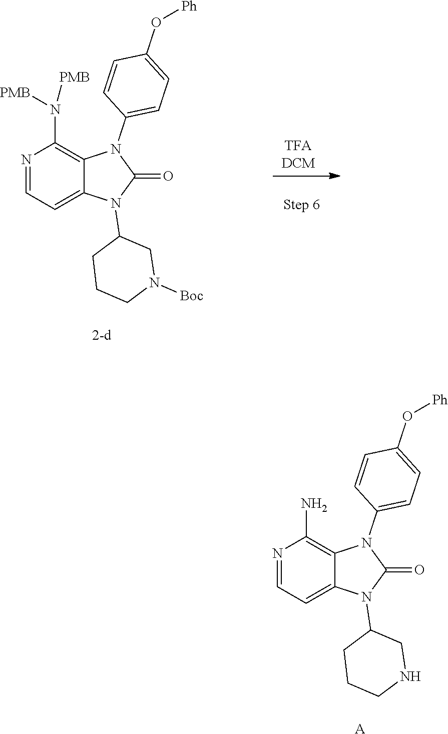

A solution of compound 2-d (6.0 g, purity 70.8%, 5.7 mmol) in trifluoro acetic acid (12 mL, 27.4 eq) under an inert atmosphere of nitrogen was stirred 36 h at ambient temperature to reach full conversion by HPLC. 10V of a satured aq. NaHCO₃ solution was added. The reaction mixture was extracted with 3*10V DCM. The organic phases were combined and washed with 2*10V of a satured aq. NaHCO₃ solution then washed with 10V deionized water and concentrated by rotary evaporation to afford compound A as a brown oil.

NMR data was obtained as described in FIG. 7.

LCMS data was consistent with compound A.

### Example S2.7 (R)-1-(1-acryloylpiperidin-3-yl)-4-amino-3-(4-phenoxyphenyl)-1,3-dihydro-2H-imidazo[4,5-c]pyridin-2-one ((I))

The compound of Formula (I) was prepared following the procedure described in Example B. It could also be prepared according to the procedure described in Example A.

NMR and LCMS data were consistent with compound (I).

### Example 3

### General Route 3

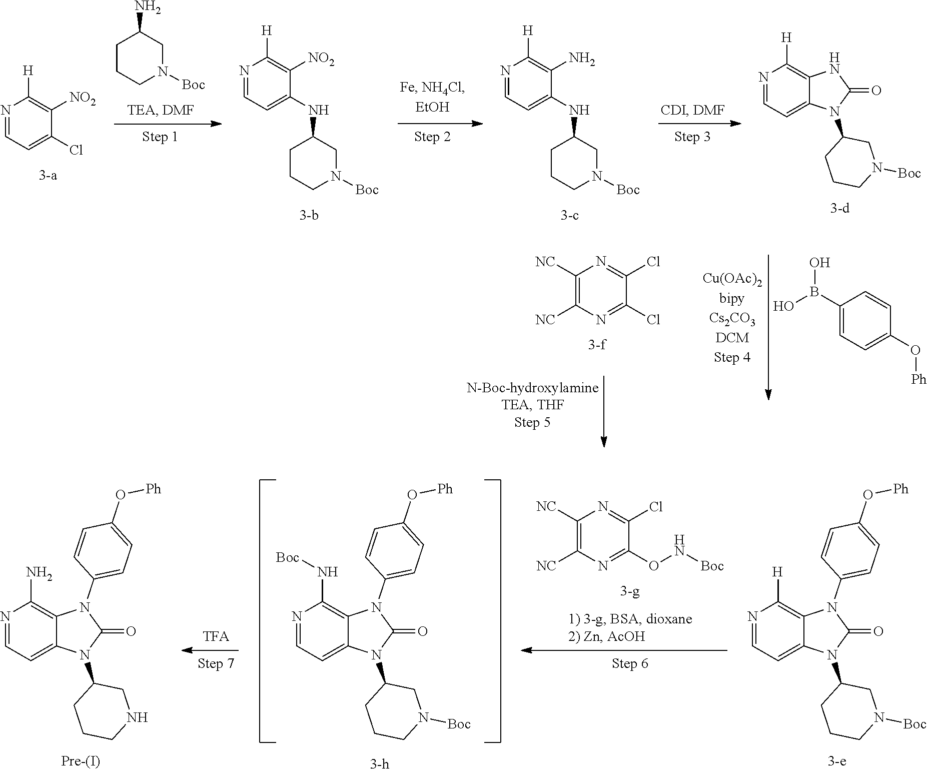

### General Route B-Example B

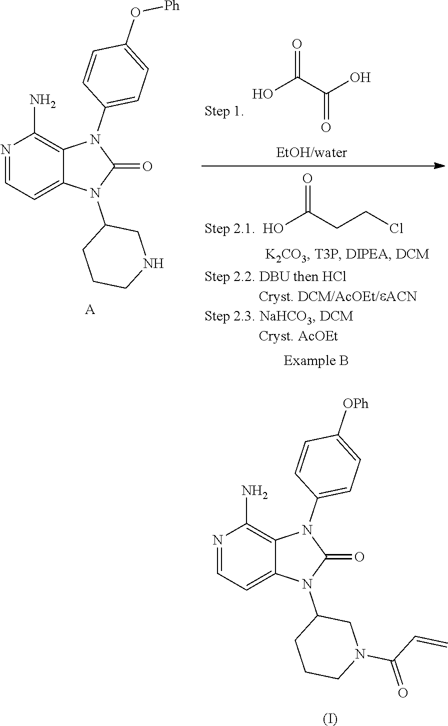

### Example S3.1 tert-butyl (R)-3-((3-nitropyridin-4-yl)amino)piperidine-1-carboxylate (3-b)

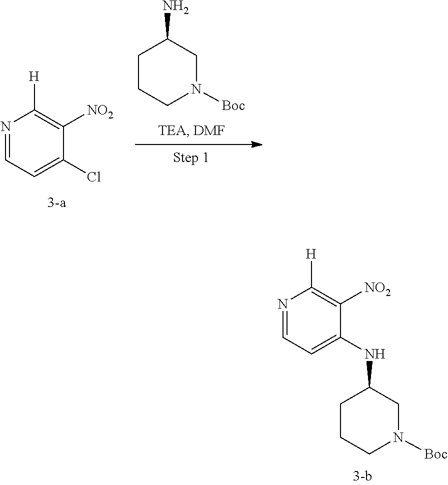 [SMILES: O=[N+]([O-])c1cnccc1Cl ; CC(C)(C)OC(=O)N1CCC[C@@H](N)C1 ; CC(C)(C)OC(=O)N1CCC[C@@H](Nc2ccncc2[N+](=O)[O-])C1]

To a solution of compound 3-a (40.0 g, 252 mmol, 1.00 eq) in DMF (250 ml) was added Et₃N (38.3 g, 378 mmol, 52.7 mL, 1.50 eq) at 0° C. under N₂. The reaction was stirred at 0° C. for 10 mins. A solution of tert-butyl (R)-3-aminopiperidine-1-carboxylate (50.5 g, 252 mmol, 1.00 eq) in DMF (250 mL) was added to the above solution dropwise at 0° C. The reaction was stirred at 20° C. for 16 h. LC-MS showed the raw material was consumed completely and one peak with desired m/z was detected. Two reactions were combined for workup. The residue was poured into water (800 mL). The aqueous phase was extracted with ethyl acetate (300 mL*2). The combined organic phase was washed with brine (300 mL), dried with anhydrous Na₂SO₄, filtered and concentrated under vacuum. Compound 3-b (120 g, 349 mmol, 69.2% yield, 93.8% purity) was obtained as yellow oil.

LCMS: RT =0.715 min, M+1=323.2.

¹H NMR (400 MHZ, CDCl₃) δ 9.22 (s, 1H), 8.30 (d, J=6.0 Hz, 1H), 8.20 (d, J=6.8 Hz, 1H), 6.81 (s, 2H), 3.91. (s, 1H), 3.61-3.66 (m, 2H), 3.04-3.25 (m, 2H), 2.05-2.07 (m, 1H), 1.74-1.84 (m, 2H), 1.45-1.47 (m, 1H), 1.43 (s, 9H).

### Example S3.2 tert-butyl (R)-3-((3-aminopyridin-4-yl)amino)piperidine-1-carboxylate (3-c)

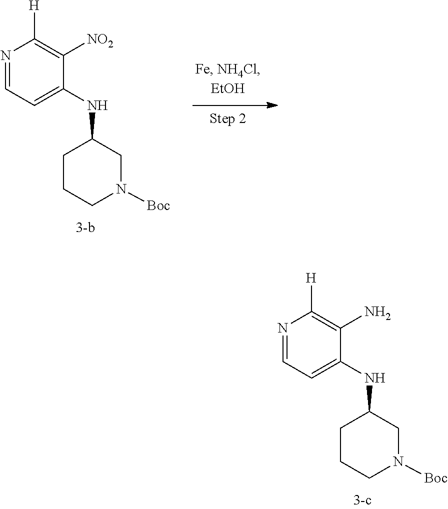 [SMILES: CC(C)(C)OC(=O)N1CCC[C@@H](Nc2ccncc2[N+](=O)[O-])C1 ; CC(C)(C)OC(=O)N1CCC[C@@H](Nc2ccncc2N)C1]

To a solution of compound 3-b (60.0 g, 186 mmol, 1.00 eq) in EtOH (350 mL) was added Fe (51.9 g, 930 mmol, 5.00 eq) at 35° C. under N₂. The reaction was stirred at 35° C. for 10 mins. Then, a solution of NH₄Cl (49.8 g, 930 mmol, 5.00 eq) in H₂O (100 mL) was poured into the above solution at 35° C., and the mixture was heated at 60° C. for 13 h. TLC (dichloromethane/methanol=5/1, R_{f}=0.50) indicated the raw material was consumed completely. Two reactions were combined for workup. The residue was filtered on celite and washed with EtOH (3000 mL). The filtrate was concentrated under vacuum. The solution was poured into water (1000 mL). The aqueous phase was extracted with DCM (500 mL*3). The combined organic phase was washed with brine (300 mL), dried with anhydrous Na₂SO₄, filtered and concentrated under vacuum. The residue was purified by column chromatography (SiO₂, dichloromethane/methanol=50/1 to 0/1). Compound 3-c (40.0 g, 135 mmol, 36.3% yield, 98.9% purity) was obtained as a light brown solid.

LCMS: RT=1.775 min, M+1=293.1.

¹H NMR (400 MHZ, CDCl₃) δ 9.22 (s, 1H), 8.30 (d, J=6.0 Hz, 1H), 8.20 (d, J=6.8 Hz, 1H), 6.81 (s, 2H), 3.91. (s, 1H), 3.61-3.66 (m, 2H), 3.04-3.25 (m, 2H), 2.05-2.07 (m, 1H), 1.74-1.84 (m, 2H), 1.45-1.47 (m, 1H), 1.43 (s, 9H).

### Example S3.3 tert-butyl (R)-3-(2-oxo-2,3-dihydro-1H-imidazo[4,5-c]pyridin-1-yl)piperidine-1-carboxylate (3-d)

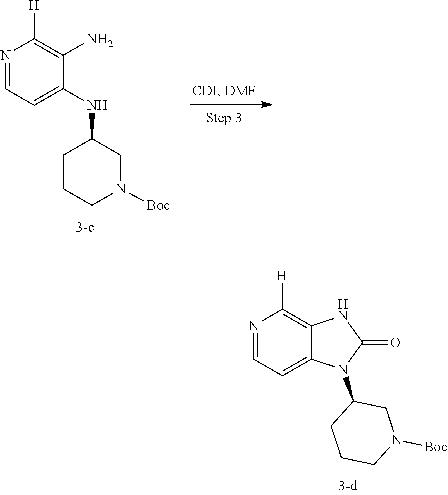 [SMILES: CC(C)(C)OC(=O)N1CCC[C@@H](Nc2ccncc2N)C1 ; CC(C)(C)OC(=O)N1CCC[C@@H](n2c(=O)[nH]c3cnccc32)C1]

To a solution of compound 3-c (20.0 g, 68.4 mmol, 1.00 eq) in DMF (100 mL) was added CDI (13.3 g, 82.1 mmol, 1.20 eq) at 20° C. The mixture was stirred at 20° C. for 2 h. TLC (dichloromethane/methanol=5/1, R_{f}=0.05) indicated the raw material was consumed completely. The solution was poured into water (500 mL). The aqueous phase was extracted with DCM (80.0 mL*3). The combined organic phase was washed with brine (50.0 mL), dried with anhydrous Na₂SO₄, filtered and concentrated under vacuum. The residue was purified by column chromatography (SiO₂, dichloromethane/methanol=50/1 to 0/1). Compound 3-d (12.0 g, 37.7 mmol, 55.1% yield, 100% purity) was obtained as a light yellow solid.

LCMS: RT=1.753 min, M+1=319.1.

¹H NMR (400 MHZ, CDCl₃) δ 8.40 (s, 1H), 8.30 (d, J=5.2 Hz, 1H), 7.07 (d, J=5.6 Hz, 1H), 4.10-4.22 (m, 3H), 3.40-3.46 (m, 1H), 2.77 (s, 1H), 2.33-2.41 (m, 1H), 2.00-2.03 (m, 1H), 1.86-1.90 (m, 1H), 1.62-1.70 (m, 1H), 1.46 (s, 9H)

As shown in the Appendix, this step could also be achieved using H₂ and Pd/C in ethyl acetate, followed by (Boc)₂O and DMAP.

### Example S3.4 tert-butyl (R)-3-(2-oxo-3-(4-phenoxyphenyl)-2,3-dihydro-1H-imidazo[4,5-c]pyridin-1-yl)piperidine-1-carboxylate (3-e)

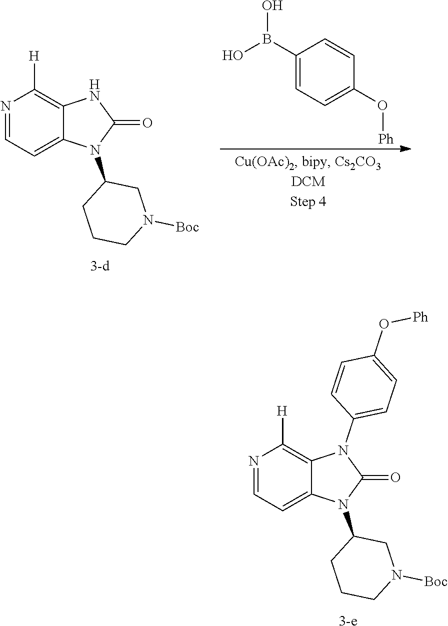

Compound 3-d (9.80 g, 30.78 mmol, 1.00 eq), copper (II) acetate (2.8 g, 15.39 mmol, 0.5 eq), 2,2′-bipyridine (2.4 g, 15.39 mmol, 0.5 eq), cesium carbonate (20.06 g, 61.56 mmol, 2 eq) were added to DCM (196 mL). The mixture was stirred at 20° C. for 1 h under atmosphere and air flow. Then, 4-phenoxyphenylboronic acid (14.82 g, 69.26 mmol, 2.25 eq) was added. After a night, LCMS indicated that 13.8% of starting material remained. 34 g (117.7 mmol, 3.4 eq) of 4-phenoxyphenylboronic acid were added by portions with copper (II) acetate (0.7 g, 3.85 mmol, 0.125 eq) to the mixture for completing the reaction after two days. DCM (196 mL) and water (98 mL) were added to the mixture then the organic layer was extracted and washed with water (98 mL). The organic layer was concentrated under reduced pressure to give a residue (40 g). This residue was purified by column chromatography (SiO₂, DCM/MeOH=100/0 to 98/2). Compound 3-e (9 g, 18.49 mmol, 60% yield) was obtained as a clear brown solid.

NMR and LCMS data were consistent with compound 3-e.

### Example S3.5 tert-butyl ((3-chloro-5,6-dicyanopyrazin-2-yl)oxy)carbamate (3-g)

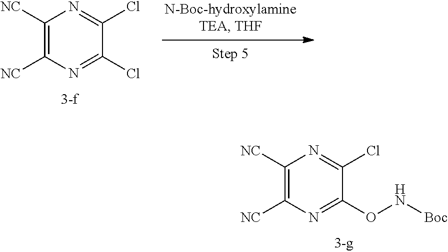 [SMILES: N#Cc1nc(Cl)c(Cl)nc1C#N ; CC(C)(C)OC(=O)NOc1nc(C#N)c(C#N)nc1Cl]

To a solution of compound 3-f (220.8 g, 1.66 mol, 1.1 eq) N-Boc-hydroxylamine (300.0 g, 1.51 mol, 1.0 eq) in THF (3.0 L) was added Et₃N (153.0 g, 1.51 mol, 1.0 eq) at −20° C. dropwise over 30 mins. The mixture was stirred at −20° C. for 10 mins. TLC (Petroleum ether/Ethyl acetate=3/1, R_{f} (product)=0.46) indicated starting material was consumed completely. The two suspensions were filtered, the filtrate was concentrated under vacuum to give a residue. The residue was purified by column chromatography (SiO₂, Petroleum ether/Ethyl acetate=50/1 to 0/1). Compound 3-g (500 g, 1.42 mol, 47.11% yield, 84% purity) was obtained as yellow solid. The solid was purified by column chromatography on silica gel (100-200 mesh, petroleum ether/ethyl acetate=50/1 to 0/1) again. The crude product was purified by re-crystallization from a solution of ethyl actate and petroleum ether (3V, 1/5) at 50° C. to give compound 3-g (205 g, 682 mmol, 40.3% yield, 98.3% purity) as a white solid.

LCMS: RT=3.358 min, M+23=318.0.

¹H NMR (400 MHZ, CDCl₃) δ 8.19 (s, 1H), 1.54 (s, 9H).

### Example S3.6 tert-butyl (R)-3-(4-((tert-butoxycarbonyl)amino)-2-oxo-3-(4-phenoxyphenyl)-2,3-dihydro-1H-imidazo[4,5-c]pyridin-1-yl)piperidine-1-carboxylate (3-h)

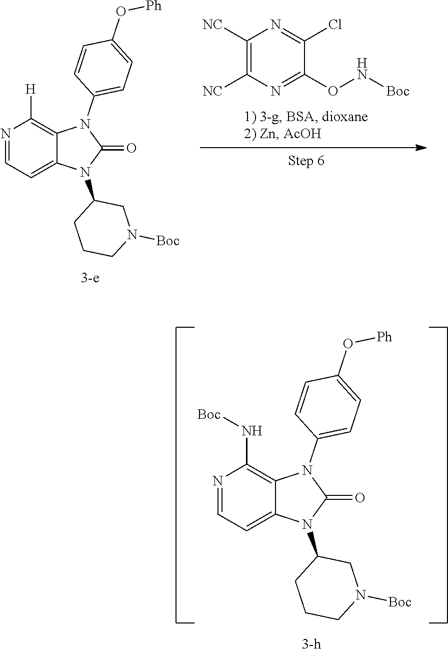

Anhydrous 1,4-dioxane (90 ml) was added to compound 3-e (3 g, 6.17 mmol, 1 eq) and N,O-Bis(trimethylsilyl)acetamide (3.76 g, 18.5 mmol, 3 eq). The mixture was stirred at RT under N2 atmosphere and then compound 3-g (2.73 g, 9.25 mmol, 1.5 eq) was added. The mixture was warmed at 80° C. for 8 h during which compound 3-g (2.08 g, 7.05 mmol, 0.875 eq) was added by portions. LCMS indicated that 4.7% of starting material remained after a night. The reaction mixture was cooled at RT then Zn (2.02 g, 30.83 mmol, 5 eq) and glacial acetic acid (90 ml) were added. After 2 h at RT, the mixture was filtrated through celite.

NMR and LCMS data were consistent with compound 3-h.

### Example S3.7 (R)-4-amino-3-(4-phenoxyphenyl)-1-(piperidin-3-yl)-1,3-dihydro-2H-imidazo[4,5-c]pyridin-2-one (A)

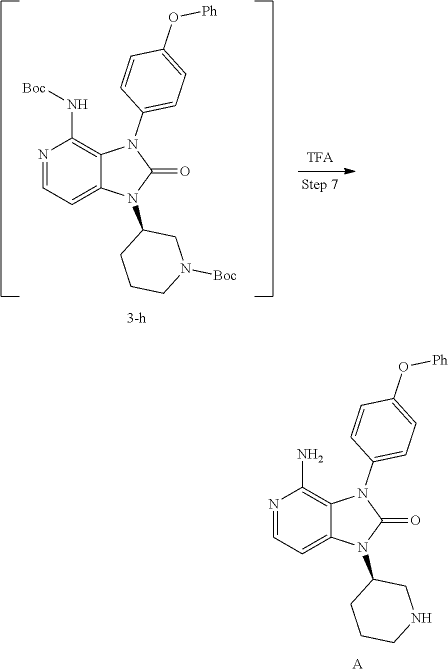

To the resulting filtration liquors was added TFA (15 mL) to complete the partial deprotection that already began to occur. The reaction mixture was warmed at 80° C. during 10 h. LCMS indicated that acetamide derivatives were generated alongside desired product. Thereby, KOH/EtOH (1 M, 45 mL) was added to the mixture and warmed at 70° C. (overnight and 8 h) to recover 2-amino fuction from corresponding acetamido impurities. The mixture was filtrated through celite in order to eliminate some tarry material. The resulting solution was concentrated. DCM (90 mL) and water (60 mL) were poured on the dry residue and the resulting organic layer was separated. Aqueous layer was re-extracted with DCM (30 mL) and the combined organic layers were washed twice with water (2*30 mL). An HPLC title enabled to estimate 17% of yield in the organic layer. The organic layer was concentrated under reduced pressure to give a residue. The residue was purified twice by column chromatography (SiO₂, DCM/MeOH=100/0 to 75/25). Compound A (60 mg, p-87%, 0.13 mmol, 2.4% yield) was obtained as a clear brown solid.

LCMS data was consistent with compound A.

NMR data obtained was consistent with the spectrum shown in FIG. 7.

### Example S3.7 (R)-1-(1-acryloylpiperidin-3-yl)-4-amino-3-(4-phenoxyphenyl)-1,3-dihydro-2H-imidazo[4,5-c]pyridin-2-one (I)

The compound of Formula (I) was prepared following the procedure described in Example B. It could also be prepared according to the procedure described in Example A.

NMR and LCMS data were consistent with compound (I).

### Final Route-Example A

### General Route A

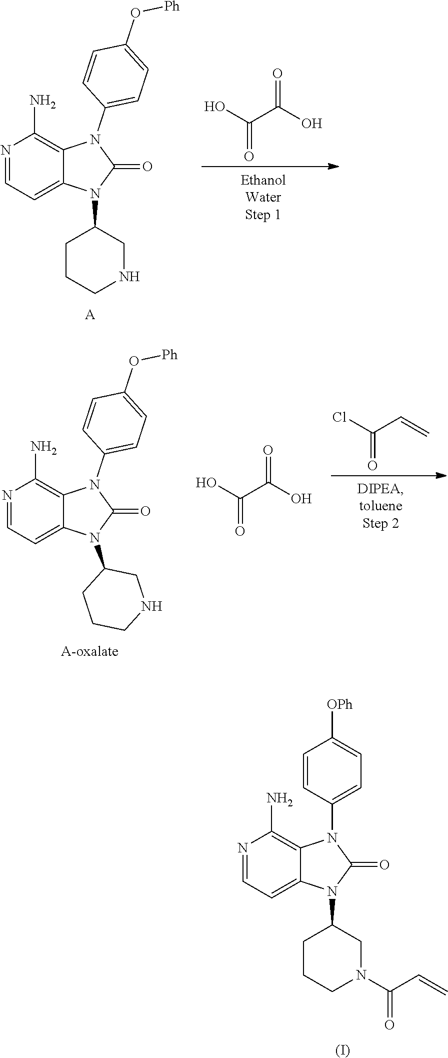

### Example SA.1 (R)-4-amino-3-(4-phenoxyphenyl)-1-(piperidin-3-yl)-1,3-dihydro-2H-imidazo[4,5-c]pyridin-2-one (A-oxalate)

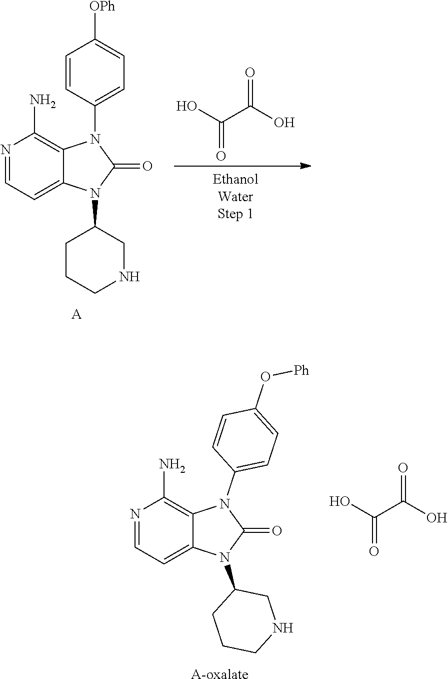

To compound A dissolved with 16V EtOH was added a solution of oxalic acid (655 mg, 1.3 eq) in 4V EtOH. The solution was stirred 2 h at 15° C. and the solid was filtered off. The cake was washed with 2V EtOH then 2V MTBE. 2.7 g of A-oxalate was obtained as a grey solid (85% purity, 81.5% corrected yield). A second jet was filtered off 680 mg (43.5% purity, 10.4% corrected yield).

NMR and LCMS data were consistent with compound A-oxalate.

### Example SA.2 (R)-1-(1-acryloylpiperidin-3-yl)-4-amino-3-(4-phenoxyphenyl)-1,3-dihydro-2H-imidazo[4,5-c]pyridin-2-one ((I))

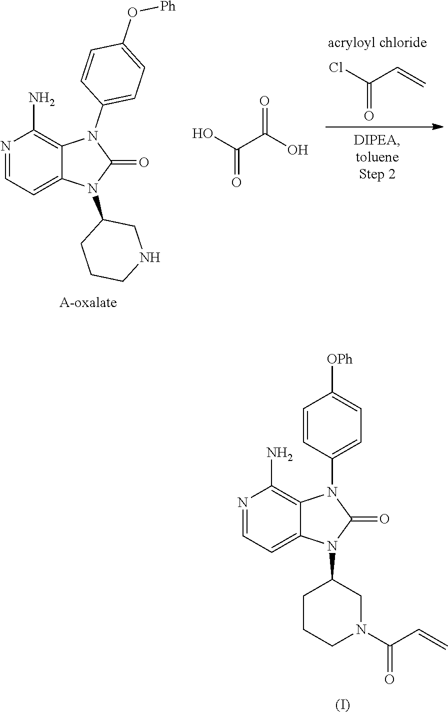

Compound (I) can be made similar to as disclosed in U.S. Pat. No. 9,688,676B2. For example, compound (I) can be prepared by reacting compound A-oxalate with acryloyl chloride, optionally using a DIPEA base in toluene instead of TEA in DCM/MeOH as described in U.S. Pat. No. 9,688,676B2.

NMR and LCMS data were consistent with compound (I).

### Final Route-Example B

### General Route B.

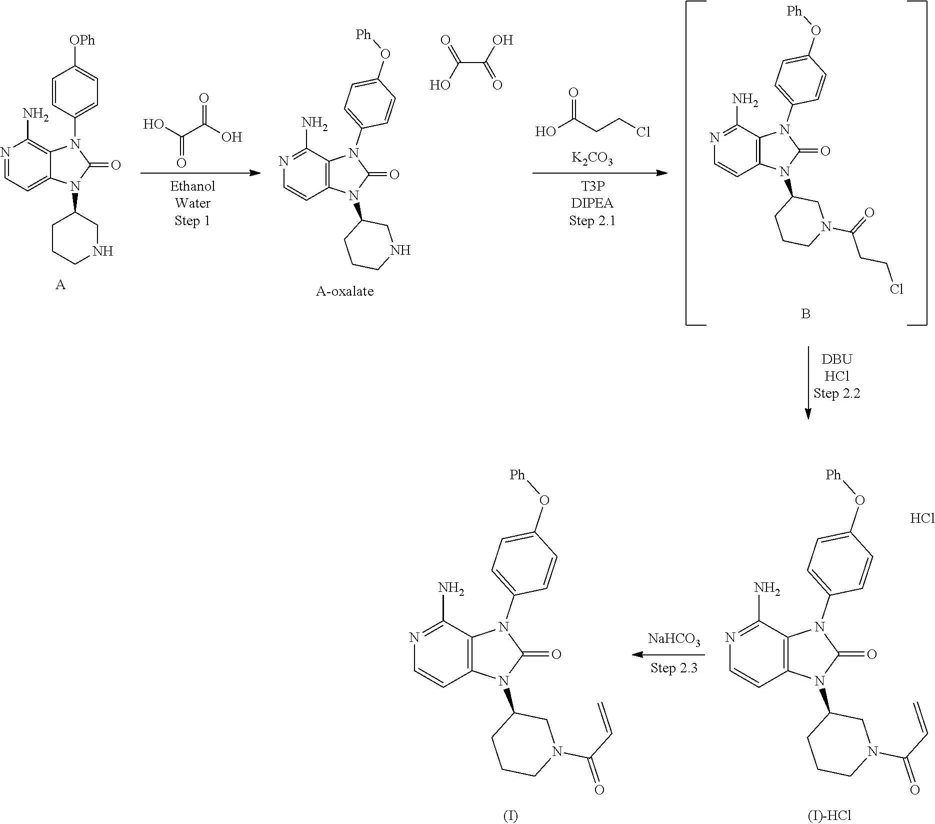

### Example SB.1 (R)-4-amino-3-(4-phenoxyphenyl)-1-(piperidin-3-yl)-1,3-dihydro-2H-imidazo[4,5-c]pyridin-2-one (A-oxalate)

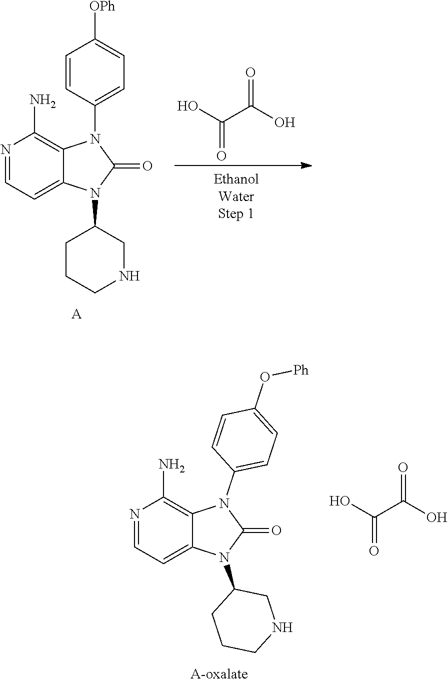

To compound A dissolved with 16V EtOH was added a solution of oxalic acid (655 mg, 1.3 eq) in 4V EtOH. The solution was stirred 2 h at 15° C. and the solid was filtered off. The cake was washed with 2V EtOH then 2V MTBE. 2.7 g of A-oxalate was obtained as a grey solid (85% purity, 81.5% corrected yield). A second jet was filtered off 680 mg (43.5% purity, 10.4% corrected yield).

NMR and LCMS data were consistent with compound A-oxalate.

### Example SB.2 (R)-1-(1-acryloylpiperidin-3-yl)-4-amino-3-(4-phenoxyphenyl)-1,3-dihydro-2H-imidazo[4,5-c]pyridin-2-one (I)

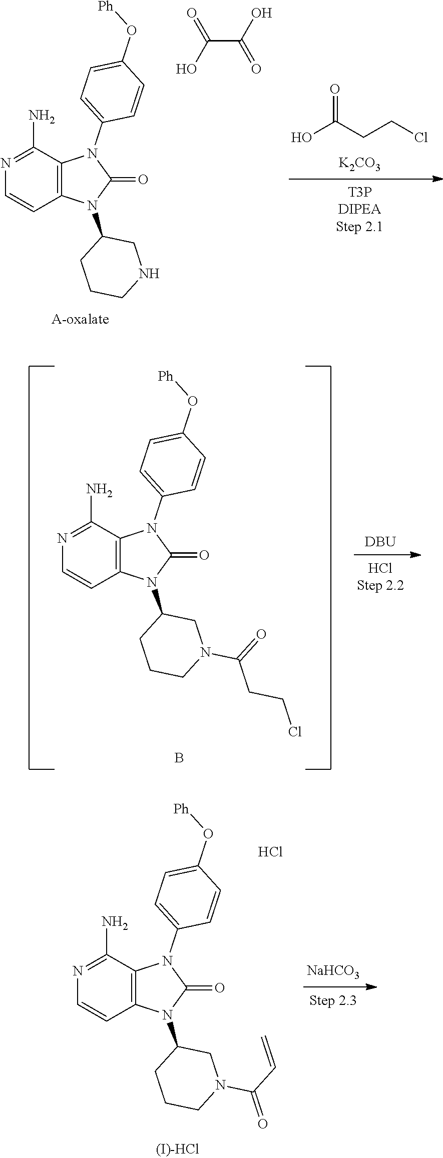

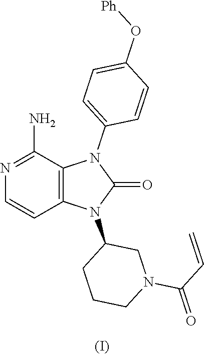

Purified water (7.5 vol.) and K₂CO₃ (at least 3.0 eq.) was added to compound A-oxalate (hydrate; corresponds to 1 eq. compound A in DCM (12 vol.) at 20° C., and the reaction mixture was stirred for at least 2 h. The reaction mixture was then allowed to settle and separate. The organic layers were collected and washed 1-2 times with water (7.5 vol.) to afford A-oxalate in DCM solution. The solution was concentrated to 12 vol. and mixed with DIPEA (4 eq.) at 20° C. Next, a solution of 3-chloropropanoic acid (1.05 eq.) in DCM (2.3 vol.) and T3P (50% DCM solution, 1 eq.) was added at 20° C. Compound B was formed in situ. Next, DBU (4 eq.) was added to the reaction mixture at 30° C. over at least 30 min. and the resulting mixture was kept at 30° C. for at least 2 hr. The organic layer was washed 3-5 times with HCl (1 N, 10 vol.) at 20° C. Next, the organic layer was concentrated to 2.73 vol. and the temperature was adjusted to 35° C. Compound (I)-HCl seeds (0.1 kg/kg) were added to the organic layer at 35° C. and the temperature was maintained for at least 1 hr. Ethyl acetate (2 vol.) was then added and a temperature of 35° C. was maintained for at least 1 h. Next, the reaction mixture was cooled to 10° C. and ACN (1.07 vol.) was added. The mixture was neutralized with NaHCO₃. The mixture was cooled to 0° C. A filter-dryer was charged with the resulting suspension and re-slurried with DCM (0.72 vol.)/AcOEt (0.63 vol.)/ACN (0.45 vol.) at 0° C. The solid was filtered, washed twice with AcOEt (1.8 vol.), and twice with ACN (1.8 vol.). The resulting compound of Formula (I) was dried at a maximum temperature of 50° C. The following elemental analysis was performed by Galbraith Laboratories: Carbon, Hydrogen, and Nitrogen Determination using the PerkinElmer2400 Series II CHNS/O Analyzer and determination of Total Halogens or Total Halides by Potentiometric Titration.

NMR and LCMS data were consistent with compound (I).

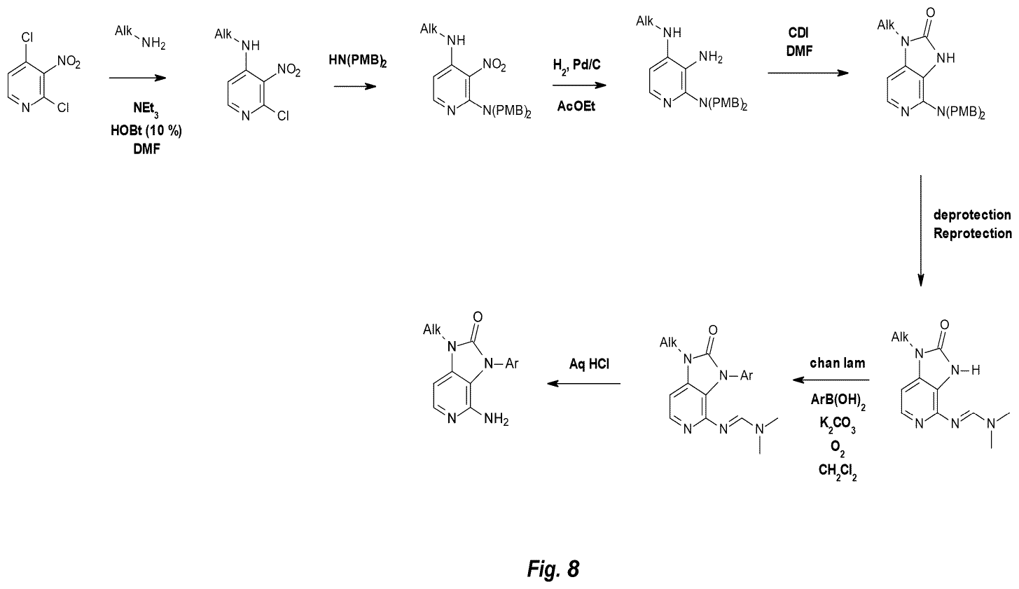

Additional Studies: Additional studies were conducted to improve the 10-step linear synthesis for preparing the 2,3,4-aminopyridine ring system as described in FIG. 8. This 10-step linear synthesis had an overall 20% yield, several steps that contain undesirable solvents, and Chan Lam coupling step which was a challenge to scale-up. Therefore, although developmental studies successfully validated the industrial scale of this process, an improved synthesis route was in need. The objective was to develop a more efficient and environmentally friendly process as the negative environmental impact of solvents such as DMF-DMA, DMF, and CH₂Cl₂ were considered.

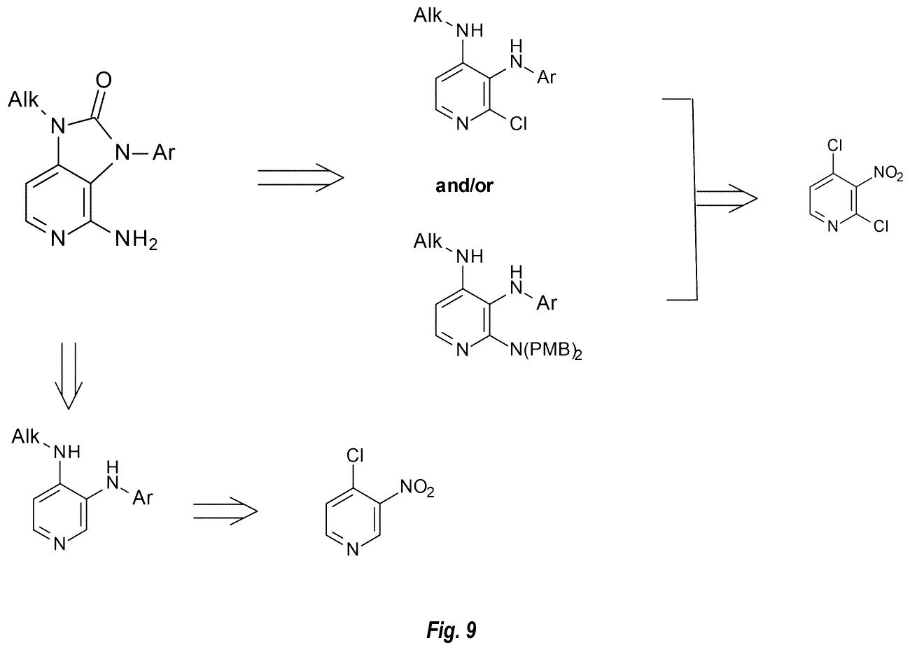

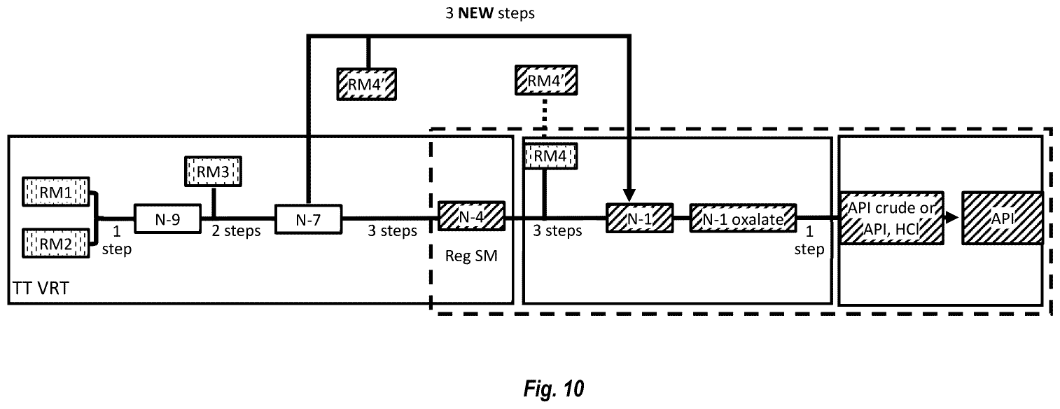

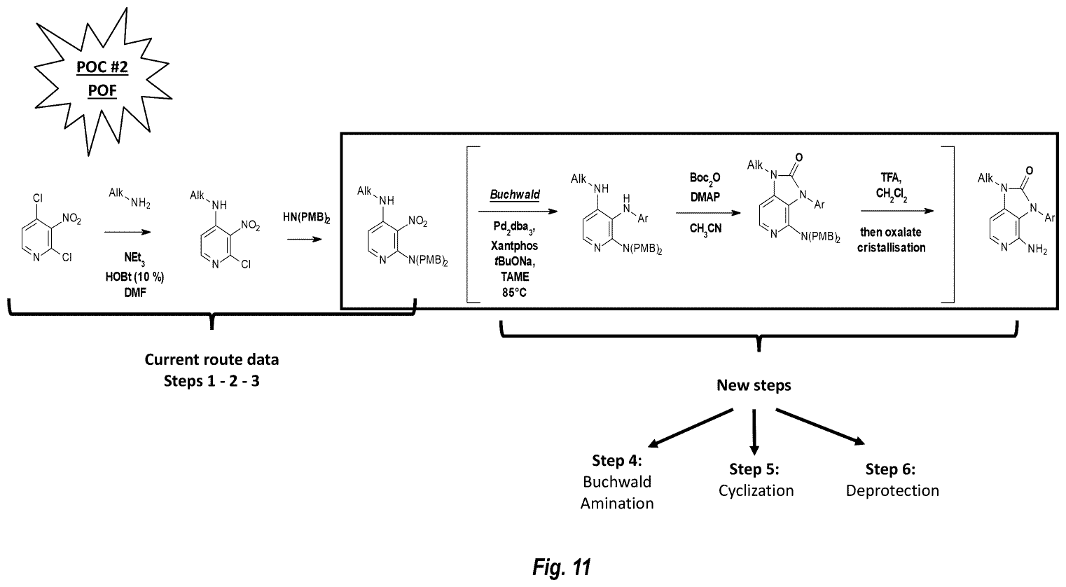

Accordingly, a restrosynthetic scheme, as described in FIG. 9, was prepared to identify routes to improve. Based on this scheme, a second generation route was devised, which bridged the N-1 intermediate to the N-7 intermediate of the current process, resulting in a 6 step synthesis as shown in FIG. 10. The second generation route obviated the Chan Lam coupling step and instead introduced 3 new steps as described in FIG. 11: a Buchwald reaction, cyclization reaction, and deprotection reaction.

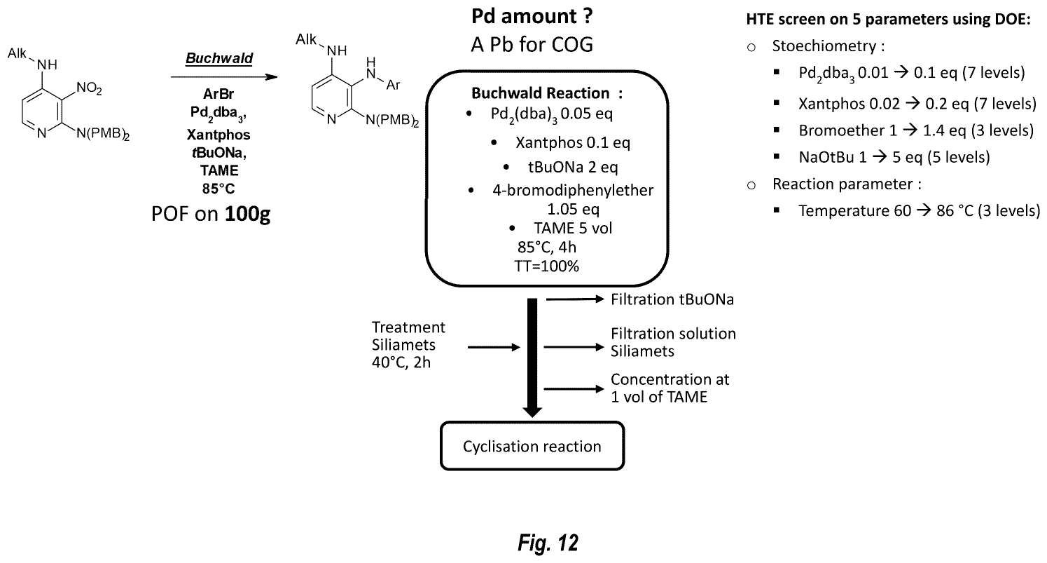

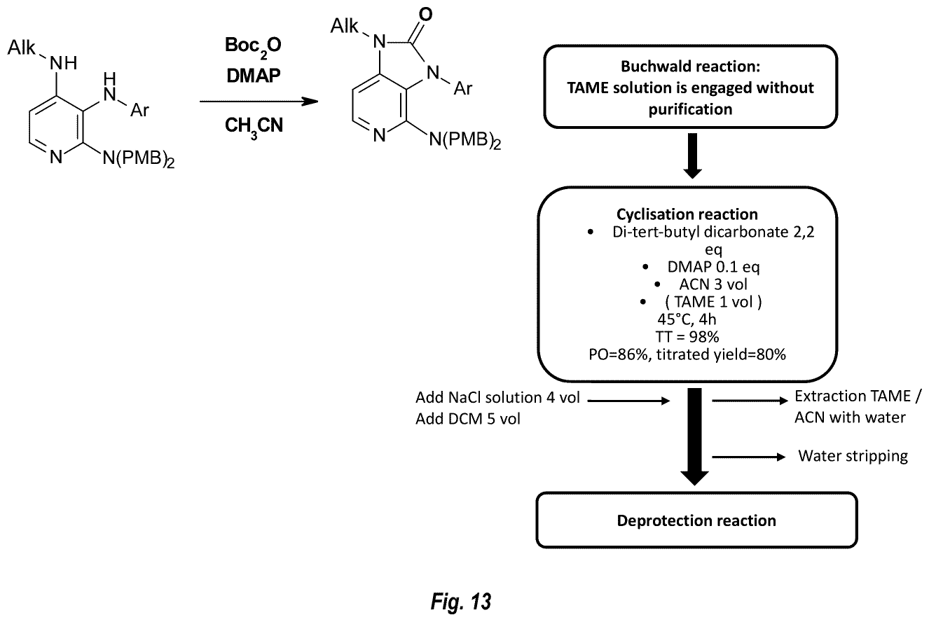

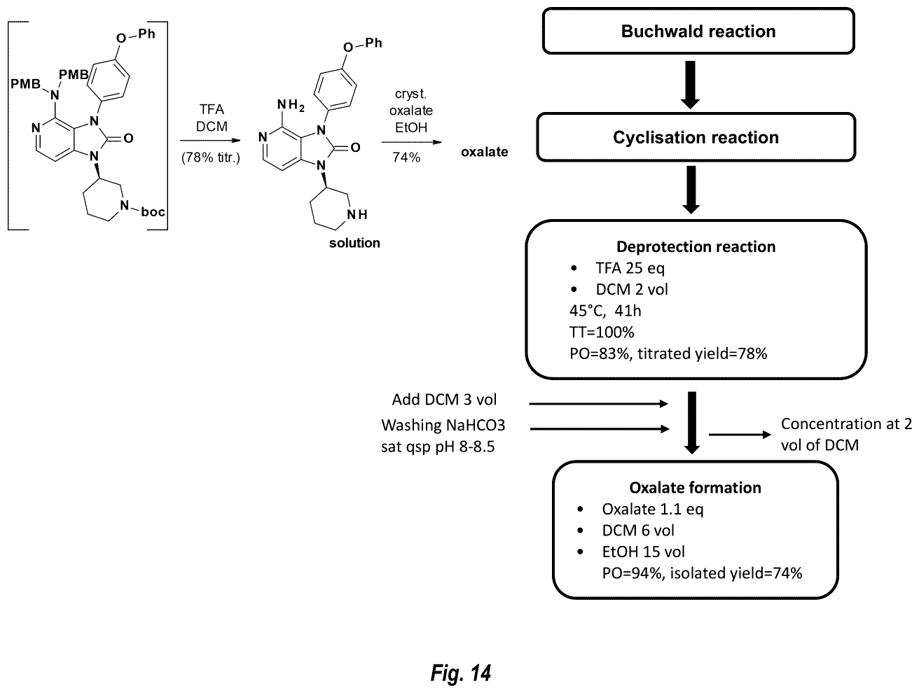

The proof of feasability (POF) process of the Buchwald reaction was explored on a 100 g scale as described in FIG. 12. Various reaction conditions were tested using design of experiments (DOE) screening. The cyclization and deprotection reactions were also explored through a proof of concept process as described in FIG. 13 and FIG. 14.

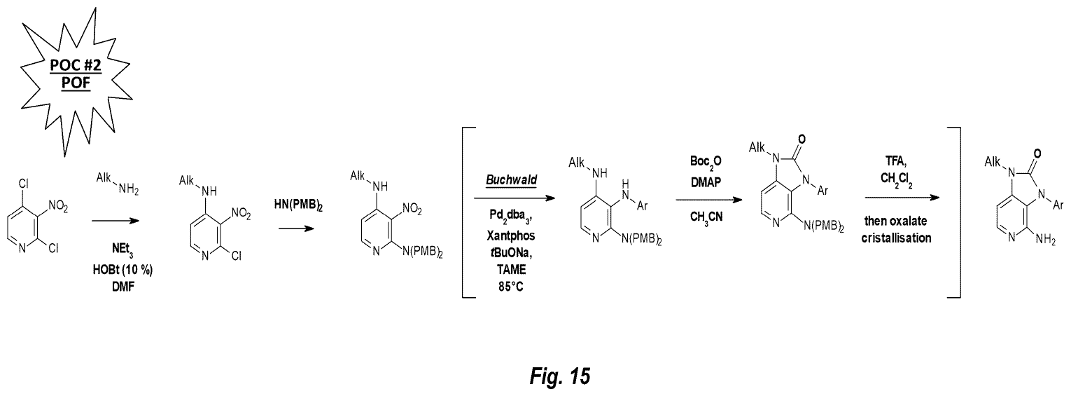

Eco-friendly approaches were explored for the new synthesis route as described in FIG. 15. The 3 steps in the new route were allowed to proceed consecutively without purification of the intermediates. The Buchwald reaction used drastically reduced % of catalyst and % of ligand (1 mol % each), and the Pd catalyst could be replaced with a Cu catalyst. Instead of dichloromethane, the deprotection step could be performed in anisole, a greener solvent.

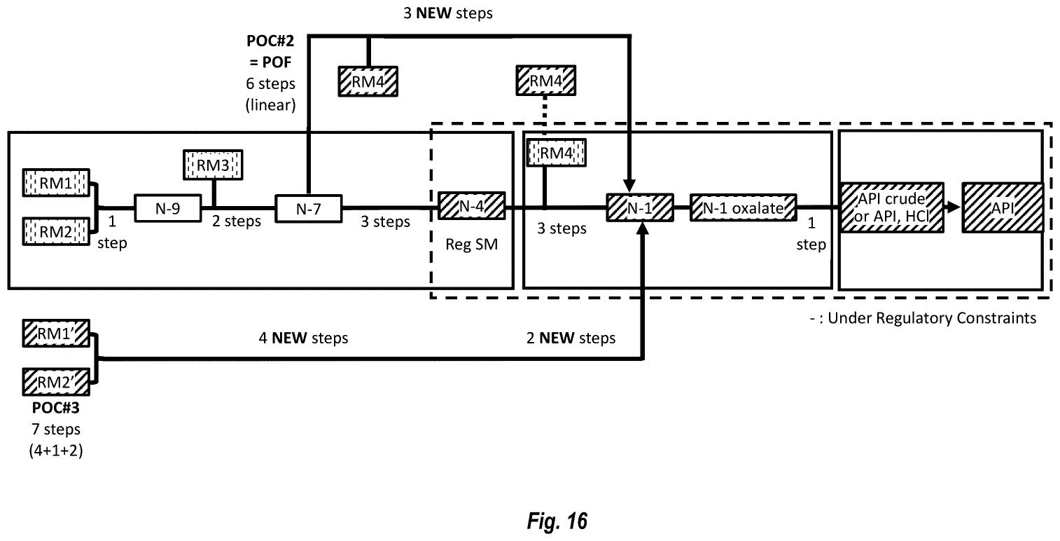

Another second generation route was devised, which prepared the N-1 intermediate through a complete new process that is more convergent and does not require palladium, as described in FIG. 16.

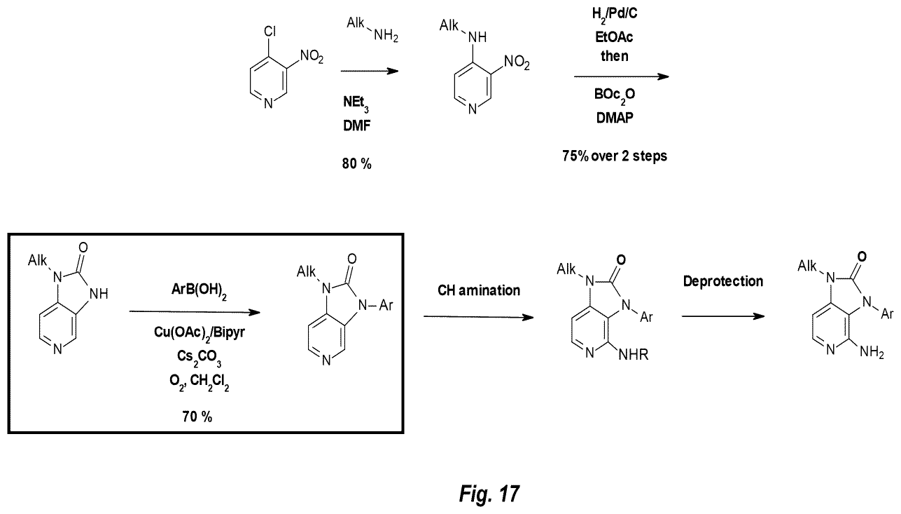

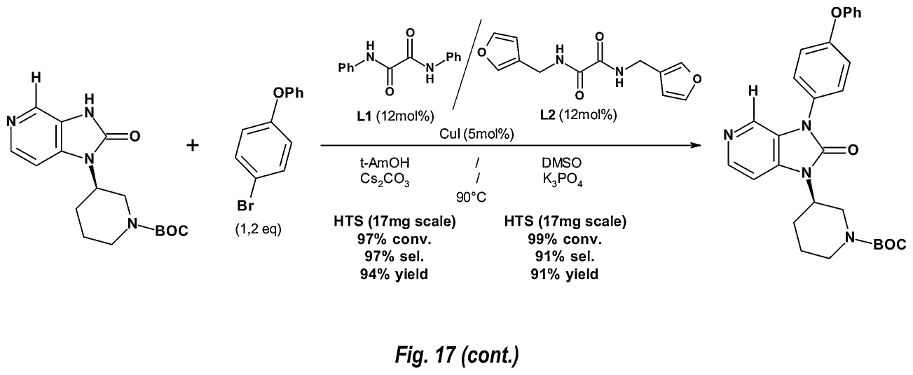

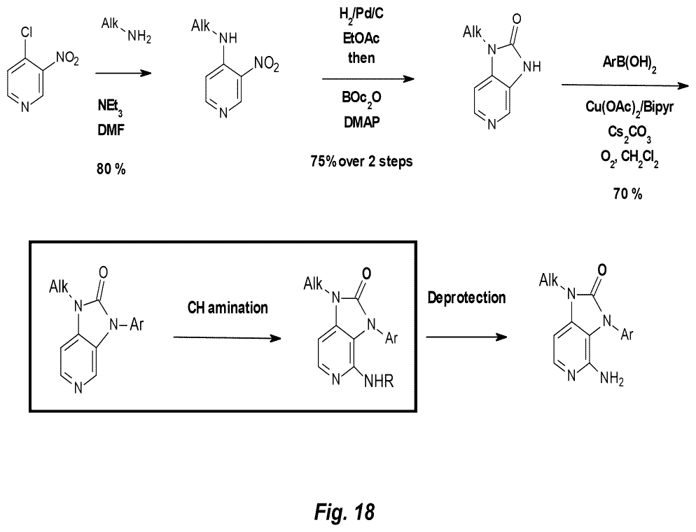

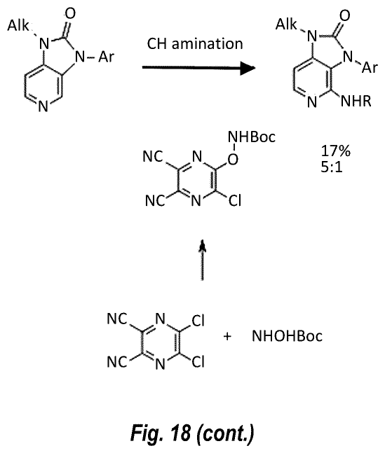

The new second generation route was able to bypass Chan Lam coupling through late stage NHBoc introduction as described in FIG. 17. High-throughput screening studies confirmed the aryl conjugation step can be achieved in high yield with a copper catalyst. The CH amination step could be carried out in a regioselective fashion by using a highly activated pyrazine residue as described in FIG. 18. Various reaction conditions were considered, wherein the NsCl or TsCl activation agent, acetonitrile solvent, and saccharin amine surrogate were selected as the greener, more environmentally friendly options.

Although the present disclosure has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

What is claimed is:

1. A method of preparing a compound of Formula (I): 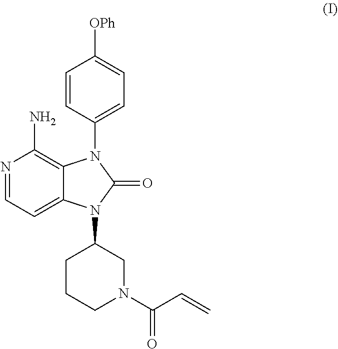 [SMILES: C=CC(=O)N1CCC[C@@H](n2c(=O)n(-c3ccc(Oc4ccccc4)cc3)c3c(N)nccc32)C1]
comprising:
reacting a compound of Formula A-oxalate: 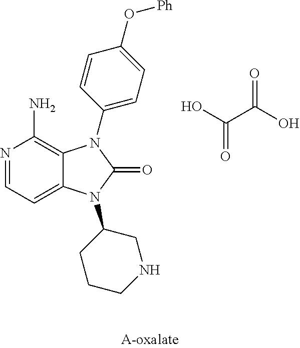 [SMILES: Nc1nccc2c1n(-c1ccc(Oc3ccccc3)cc1)c(=O)n2[C@@H]1CCCNC1 ; O=C(O)C(=O)O]
to form the compound of Formula (I), wherein the reaction conditions are chosen from:
(i) reacting the compound of Formula A-oxalate with acryloyl chloride in the presence of diisopropylethylamine in toluene; or
(ii) reacting the compound of Formula A-oxalate with 3-chloropropanoic acid in the presence of potassium carbonate, diisopropylethylamine, and propylphosphonic anhydride in dichloromethane, followed by 1,8-diazabicyclo[5.4.0]undec-7-ene in hydrochloride, and finally sodium bicarbonate.

2. The method of claim 1, wherein the compound of Formula A-oxalate is prepared by reacting a compound of Formula A: 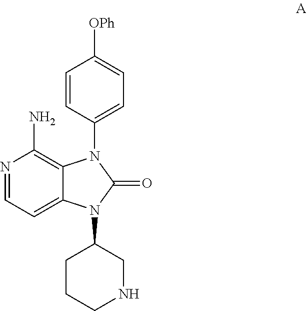 [SMILES: Nc1nccc2c1n(-c1ccc(Oc3ccccc3)cc1)c(=O)n2[C@@H]1CCCNC1]
with oxalic acid in a mixture of ethanol and water.

3. The method of any one of claim 1 or 2, wherein the compound of Formula A is prepared by reacting a compound of Formula 1-f: 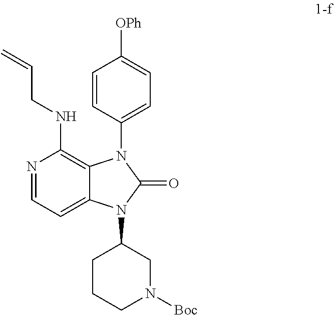 [SMILES: C=CCNc1nccc2c1n(-c1ccc(Oc3ccccc3)cc1)c(=O)n2[C@@H]1CCCN(C(=O)OC(C)(C)C)C1]
with Pd/C and methanesulfonic acid in ethanol.

4. The method of claim 3, wherein the compound of Formula 1-f is prepared by reacting a compound of Formula 1-e: 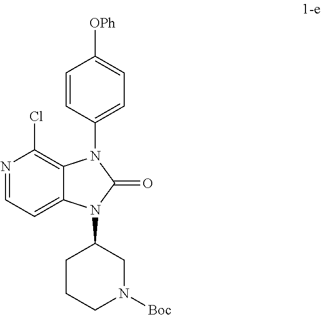 [SMILES: CC(C)(C)OC(=O)N1CCC[C@@H](n2c(=O)n(-c3ccc(Oc4ccccc4)cc3)c3c(Cl)nccc32)C1]
with allylamine in the presence of RuPhos Pd G2 and sodium tert-butoxide in dioxane.

5. The method of claim 4, wherein the compound of Formula 1-e is prepared by reacting a compound of Formula 1-d: 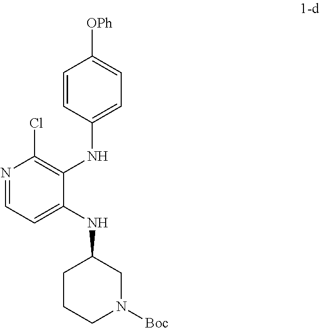 [SMILES: CC(C)(C)OC(=O)N1CCC[C@@H](Nc2ccnc(Cl)c2Nc2ccc(Oc3ccccc3)cc2)C1]
with carbonyldiimidazole in the presence of 1,8-diazabicyclo[5.4.0]undec-7-ene in toluene.

6. The method of claim 5, wherein the compound of Formula 1-d is prepared by reacting a compound of Formula 1-c: 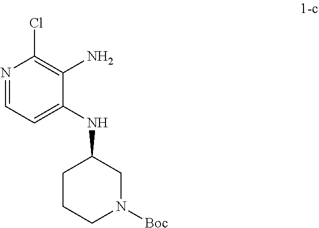 [SMILES: CC(C)(C)OC(=O)N1CCC[C@@H](Nc2ccnc(Cl)c2N)C1]
with 4-bromodiphenylether in the presence of Pd₂(dba)₃, DavePhos, and sodium tert-butoxide in toluene.

7. The method of claim 6, wherein the compound of Formula 1-c is prepared by reacting a compound of Formula 1-b: 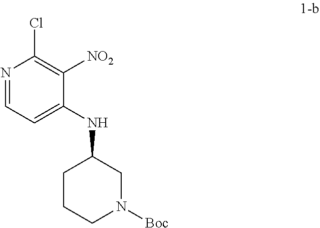 [SMILES: CC(C)(C)OC(=O)N1CCC[C@@H](Nc2ccnc(Cl)c2[N+](=O)[O-])C1]
with iron and ammonium chloride in a mixture of ethanol and water.

8. The method of claim 7, wherein the compound of Formula 1-b is prepared by reacting a compound of Formula 1-a: 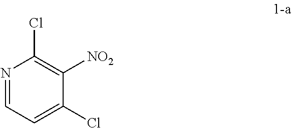 [SMILES: O=[N+]([O-])c1c(Cl)ccnc1Cl]
with tert-butyl (3R)-3-amino-piperidine-1-carboxylate in the presence of triethylamine in dimethylformamide.

9. The method of either claim 1 or 2, wherein the compound of Formula A is prepared by reacting a compound of Formula 2-d: 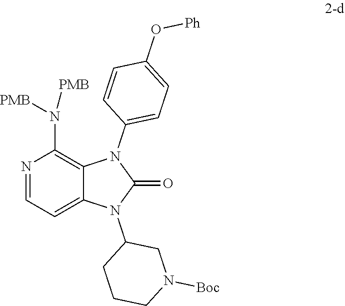
with trifluoroacetic acid in dichloromethane.

10. The method of claim 9, wherein the compound of Formula 2-d is prepared by reacting a compound of Formula 2-c: 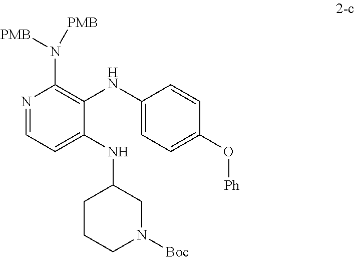
with Boc₂O in the presence of 4-dimethylaminopyridine in dimethylformamide.

11. The method of claim 10, wherein the compound of Formula 2-c is prepared by reacting a compound of Formula 2-b: 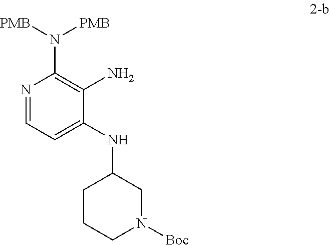
with 4-bromodiphenylether in the presence of potassium carbonate, cesium carbonate, Pd₂(dba)₃, and BrettPhos in tert-amyl methyl ether.

12. The method of claim 11, wherein the compound of Formula 2-b is prepared by reacting a compound of Formula 2-a: 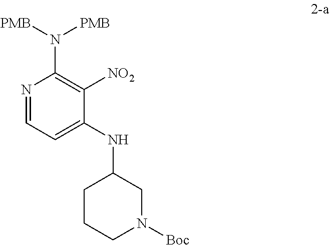
with Pd/C and H₂ in ethyl acetate.

13. The method of claim 12, wherein the compound of Formula 2-a is prepared by reacting a compound of Formula 1-b: 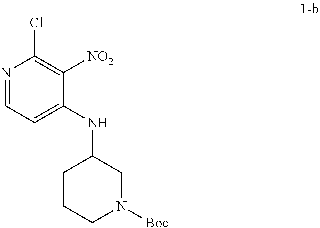 [SMILES: CC(C)(C)OC(=O)N1CCCC(Nc2ccnc(Cl)c2[N+](=O)[O-])C1]
with bis(4-methoxybenzyl)amine.

14. The method of claim 13, wherein the compound of Formula 1-b is prepared by reacting a compound of Formula 1-a: 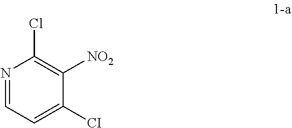
with tert-butyl (R)-3-aminopiperidine-1-carboxylate in the presence of triethylamine and HOBt in dimethylformamide.

15. The method of either claim 1 or 2, wherein the compound of Formula A is prepared by reacting a compound of Formula 3-h: 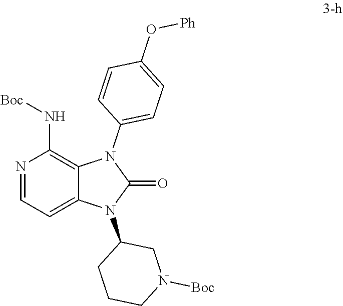 [SMILES: CC(C)(C)OC(=O)Nc1nccc2c1n(-c1ccc(Oc3ccccc3)cc1)c(=O)n2[C@@H]1CCCN(C(=O)OC(C)(C)C)C1]
with trifluoroacetic acid.

16. The method of claim 15, wherein the compound of Formula 3-h is prepared by reacting a compound of Formula 3-e: 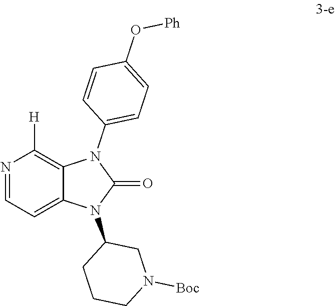 [SMILES: CC(C)(C)OC(=O)N1CCC[C@@H](n2c(=O)n(-c3ccc(Oc4ccccc4)cc3)c3cnccc32)C1]
with a compound of Formula 3-g: 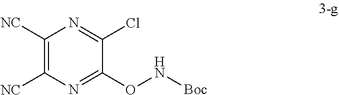 [SMILES: CC(C)(C)OC(=O)NOc1nc(C#N)c(C#N)nc1Cl]
and N,O-bis(trimethylsilyl)acetamide in dioxane, followed by zinc in acetic acid.

17. The method of claim 16, wherein the compound of Formula 3-g is prepared by reacting a compound of Formula 3-f: 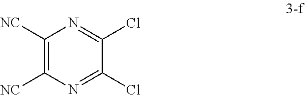 [SMILES: N#Cc1nc(Cl)c(Cl)nc1C#N]
with N-Boc-hydroxylamine in the presence of triethylamine in tetrahydrofuran.

18. The method of claim 16, wherein the compound of Formula 3-e is prepared by reacting a compound of Formula 3-d: 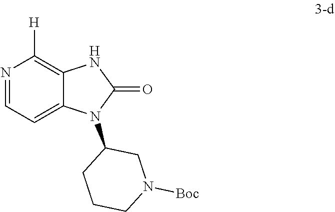 [SMILES: CC(C)(C)OC(=O)N1CCC[C@@H](n2c(=O)[nH]c3cnccc32)C1]
with 4-phenoxyphenylboronic acid in the presence of copper (II) acetate, 2,2′-bipyridine, and cesium carbonate in dichloromethane.

19. The method of claim 18, wherein the compound of Formula 3-d is prepared by reacting a compound of Formula 3-c: 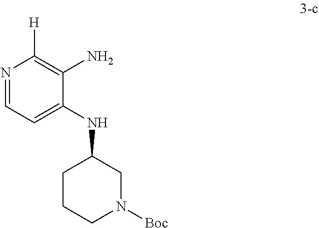 [SMILES: CC(C)(C)OC(=O)N1CCC[C@@H](Nc2ccncc2N)C1]
with carbonyldiimidazole in dimethylformamide.

20. The method of claim 19, wherein the compound of Formula 3-c is prepared by reacting a compound of Formula 3-b: 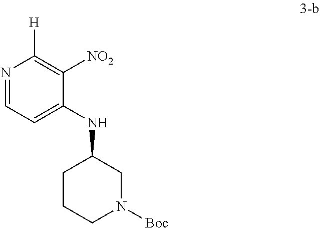 [SMILES: CC(C)(C)OC(=O)N1CCC[C@@H](Nc2ccncc2[N+](=O)[O-])C1]
with iron and ammonium chloride in ethanol.

21. The method of claim 20, wherein the compound of Formula 3-b is prepared by reacting a compound of Formula 3-a: 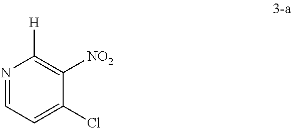
with tert-butyl (R)-3-aminopiperidine-1-carboxylate in the presence of triethylamine in dimethylformamide.

22. A compound selected from: 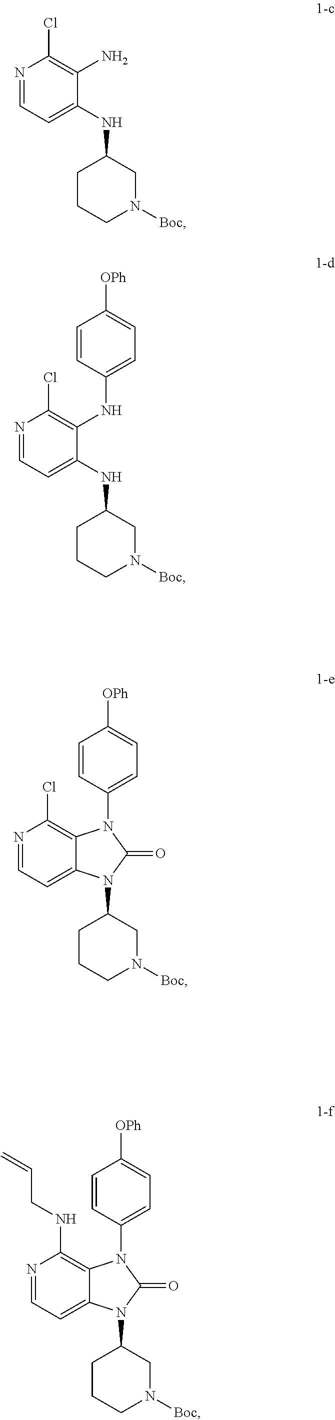 [SMILES: CC(C)(C)OC(=O)N1CCC[C@@H](Nc2ccnc(Cl)c2N)C1 ; CC(C)(C)OC(=O)N1CCC[C@@H](Nc2ccnc(Cl)c2Nc2ccc(Oc3ccccc3)cc2)C1 ; CC(C)(C)OC(=O)N1CCC[C@@H](n2c(=O)n(-c3ccc(Oc4ccccc4)cc3)c3c(Cl)nccc32)C1 ; C=CCNc1nccc2c1n(-c1ccc(Oc3ccccc3)cc1)c(=O)n2[C@@H]1CCCN(C(=O)OC(C)(C)C)C1] 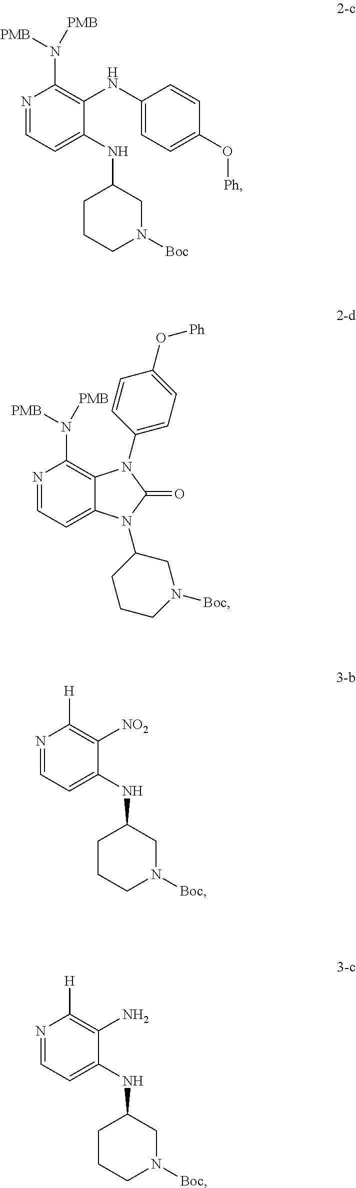 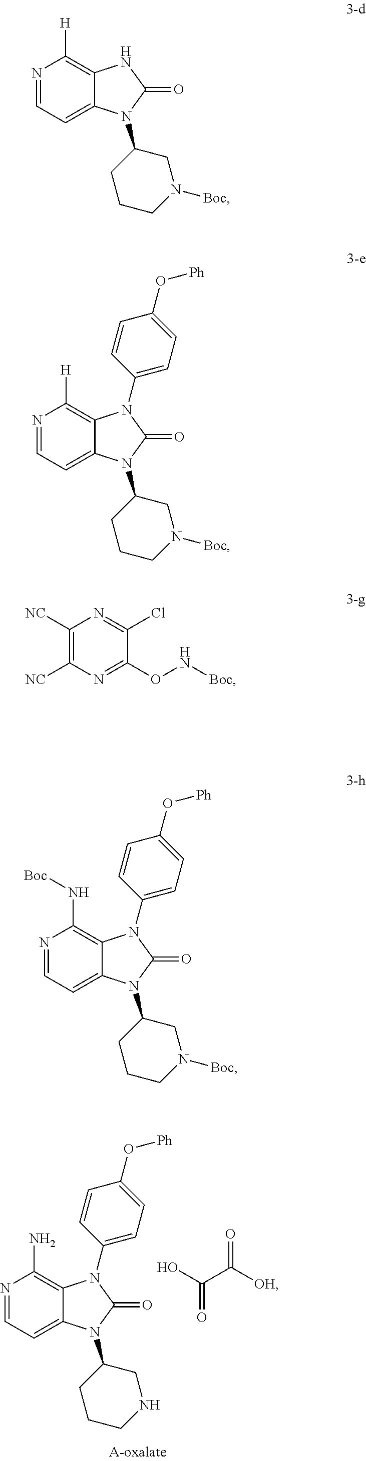
or a salt thereof.
